(19) United States
(12) Patent Application Publication
(10) Pub. No.: US 2011/0059016 A1
(43) Pub. Date: Mar. 10, 2011

(54) OPTICAL ASSAY SYSTEM WITH A MULTI-PROBE IMAGING ARRAY

(76) Inventors: Nirmala Ramanujam, Chapel Hill, NC (US); Bing Yu, Cary, NC (US); Jonathon Quincy Brown, Morrisville, NC (US)

(21) Appl. No.: 12/680,302

(22) PCT Filed: Sep. 29, 2008
(86) PCT No.: PCT/US08/78194

§ 371 Date: Nov. 23, 2010

Related U.S. Application Data
(63) Continuation-in-part of application No. 61/047,270, filed on Apr. 23, 2008.
Application No. 61/047,270 is a continuation-in-part of application No. 61/047,273, filed on Apr. 23, 2008.
(60) Provisional application No. 60/995,713, filed on Sep. 27, 2007.

(51) Int. Cl. A61K 49/00; C40B 40/06; C40B 30/04; C40B 60/12; A61P 35/00

(52) U.S. Cl. 424/9.1; 506/16; 506/9; 506/39

(57) ABSTRACT

The subject matter described herein relates to an optical assay system having a multi-probe imaging array that orients a plurality of probes with respect to one another and to a sample. According to one aspect, the subject matter described herein includes a multi-probe imaging array for contacting biological samples and simultaneously illuminating a plurality of locations on the biological sample and collecting the reflected radiation from the locations. The multi-probe imaging array can be used for the rapid imaging of biological samples, for example, during surgery.

### CROSS REFERENCE TO RELATED APPLICATIONS

This application claims the benefit of U.S. Provisional Patent Application Ser. No. 60/995,713, filed Sep. 27, 2007, U.S. Provisional Patent Application Ser. No. 61/047,273, filed Apr. 23, 2008, and U.S. Provisional Patent Application Ser. No. 61/047,270, filed Apr. 23, 2008; the disclosure of each of which is incorporated herein by reference in its entirety.

### TECHNICAL FIELD

The subject matter described herein relates to methods, compositions, and systems for detecting electromagnetic radiation reflected from or emitted by a biological sample. More particularly, the subject matter described herein relates to a multi-probe imaging array, and methods and systems for using the array to determine an indication of a physical property of the biological sample. The multi-probe imaging array can be used to illuminate multiple points on a biological sample surface simultaneously using a probe holder that holds plural illumination and detection probes in a predetermined orientation with respect to each other and with respect to the sample.

### BACKGROUND

Cancer is a significant cause of illness-related deaths in the United States. A common therapy for cancer is surgical resection of the tumor, followed by radiation, chemotherapy, or both radiation and chemotherapy. The goal of these therapies is to remove the observable tumor itself and as much additional surrounding tissue as possible in order to decrease the likelihood that pre-neoplastic or neoplastic cells that appear morphologically normal remain in the subject that can later form the basis of a recurrence or metastasis.

In many cases, however, there is a desire to limit the removal of surrounding tissue to the greatest extent possible in order to maintain the appearance and/or function of the tissue from which the tumor was resected. In order to balance the desire to remove potentially neoplastic tissue while preserving normal tissue, the surgeon will often be supported by a pathologist, who during or subsequent to the resection procedure examines the excised tissue. The pathologist's examination is designed to determine if a sufficient boundary of normal tissue surrounding the tumor has been removed to suggest that any potentially neoplastic cells have been resected. This examination of the tumor margin also can be used to determine whether further surgical intervention is necessary.

One situation where the interplay between the desire to completely remove a tumor and yet to minimize the removal of normal tissue is prominent is in breast cancer. It is estimated that about 125,000 women diagnosed with early stage breast cancer receive breast conserving surgery (BCS) each year. BCS involves removal of the cancer with a surrounding margin of normal breast tissue. An important predictor of local recurrence after BCS is pathologic margin status. Reported rates of re-excision surgery as a result of close or positive surgical margins are high (10-40%). Intraoperative frozen section and touch prep cytology have been developed to assess surgical margins and guide additional resection at the time of the initial operation. However, these techniques have not been widely adopted because of the need for specially trained personnel (pathologist), prolonged surgical time required for specimen processing (20-40 minutes), significant technical challenges, and limited coverage of the tumor margins (less than 1% of the margins are examined). Moreover, pathological margin assessment relies on visual inspection of the specimen and is unreliable for grossly occult lesions such as DCIS or invasive lobular carcinoma.

What are needed, then, are robust, reliable, and rapid strategies for assessing tumor margins. To address this need, at least in part, the subject matter described herein provides methods and systems for imaging biological samples. The subject matter described herein also addresses the more general need for an apparatus capable of rapidly illuminating a biological sample and detecting the resulting electromagnetic radiation reflected by or emitted from the sample in both in vivo and ex vivo applications for a variety of diagnostic, therapeutic, and monitoring purposes.

### SUMMARY

The subject matter described herein relates to a multi-probe imaging array for the optical imaging of biological samples. According to one aspect, the multi-probe imaging array comprises a probe holder and a plurality of electromagnetic radiation probes, wherein the probe holder orients the plurality of probes with respect to one another and with respect to a sample. In some embodiments, the probe holder comprises a series of hollow channels, wherein each of the channels surrounds a portion of a single probe and wherein the multi-probe imaging array further comprises a sample-imaging probe interface surface where an imaging tip of each of the probes is free of the probe holder such that a plurality of probes can simultaneously illuminate a plurality of locations on a biological sample with electromagnetic radiation and detect the absorption or reflection of that radiation. According to one aspect, the multi-probe imaging array can include an adapter for attaching the array to a biological sample containment and illumination apparatus in which a biological sample is present. According to another aspect, the multi-probe imaging array can be held (e.g., by hand) adjacent to a surface of a biological sample when the biological sample is present in a subject or when the biological sample has been excised from the subject to image multiple locations on the biological sample simultaneously.

The subject matter described herein also includes a method for testing a biological sample. The method includes contacting a biological sample with a multi-probe imaging array. The biological sample is illuminated through the multiple probes of the array such that each of a plurality of probes illuminate a different location on the sample in parallel. Electromagnetic radiation reemitted from the biological sample is detected. The electromagnetic radiation can be used to determine data including, but not limited to hemoglobin saturation levels, changes in response the presence of contrast agents, and the presence or absence of malignant cells.

The subject matter described herein further includes a system for imaging a biological sample. The system includes a multi-probe imaging assay, an illumination source for providing electromagnetic radiation, a detector for detecting electromagnetic radiation and converting the electromagnetic radiation into electrical signals, and a processor for determining, based on the electrical signals, an indication of a property of the biological sample. In some embodiments, the system can further comprise a biological sample containment and illumination apparatus that includes a plurality of frame members positioned with respect to each other to form an interior space for receiving a biological sample and a plurality of probe receiving locations (e.g., a plurality of apertures in the frame members).

The subject matter described herein can provide concentrations of absorbers (both endogenous and exogenous) in the biological sample and the bulk tissue scattering properties. Endogenous absorbers in biological tissue include oxygenated and deoxygenated hemoglobin, beta carotene (which is found in fatty tissues), electron carriers and structural proteins. Thus, the presently disclosed subject matter can provide two-dimensional image maps of tissue composition, metabolism, vascularity and oxygenation. The presently disclosed subject matter can also be used to image exogenous sources of absorption (organic dyes) and scattering (nanoparticles), and thus can provide the concentration and distribution of these agents in biological tissue. Thus, the presently disclosed subject matter can have utility in basic science and clinical applications, including drug discovery and assessment (in small animal models), tissue oxygenation monitoring (in reconstruction surgery, for example), assessing tumor response to chemo/radiation therapy in a variety of different sites including chest wall, cervical and head and neck cancers, intraoperative margin assessment in a variety of organ sites including the breast, brain, liver and prostate, and in epithelial cancer detection and diagnosis (skin, cervix, oral cavity, for example).

As used herein, the phrase “biological sample” includes any sample that includes biological tissue. The biological sample can be present in a subject (e.g., a human or other mammalian subject). The biological sample can further include biological tissue that has been excised from a subject. In some embodiments, a biological sample comprises a tumor biopsy, which is in some embodiments a breast tumor biopsy or a portion of a breast tumor that has been resected from a subject.

As used herein, the phrase “an indication of a property of the biological sample” refers to any property of a biological sample, including an assessment that is predictive of an area in the biological sample that has been imaged comprising all normal cells, all pre-neoplastic and/or neoplastic cells, or a combination thereof. In one implementation, the indication of the physical property may include an indication of the concentration of one or more absorbers or fluorophores or scatterer (cell nuclei) size in the biological sample.

As used herein, the term “margin” can refer to one or more of the six faces of a tumor sample (i.e., anterior, posterior, superior, inferior, medial, and lateral).

### BRIEF DESCRIPTION OF THE DRAWINGS

Preferred embodiments of the subject matter described herein will now be explained with reference to the accompanying drawings, of which:

FIG. 1 is a diagram of an exemplary system for imaging a biological sample according to an embodiment of the subject matter described herein;

FIG. 2A is a front perspective view of a multi-probe imaging array according to an embodiment of the subject matter described herein showing a sample-imaging array interface surface;

FIG. 2B is a sectional side view of the multi-probe imaging array of FIG. 2A, with probes and probe holder separated;

FIG. 2C is a sectional side view of the multi-probe imaging array of FIG. 2A with the probes present in the probe holder;

FIG. 3A is a cross-sectional view of a representative fiber optic probe bundle of the subject matter described herein showing a central core of illumination fibers (cross-hatched) surrounded by collection fibers (single-hatched) arranged in three concentric circles around the illumination fibers. Dead fibers (white) are included to form the geometry desired for the collection fibers;

FIG. 3B is a cross-sectional view of another representative fiber optic probe bundle of the subject matter described herein showing a central core of illumination fibers (cross-hatched) surrounded by collection fibers (single-hatched) arranged in three concentric circles around the illumination fibers. Dead fibers (white) are included to form the geometry desired for the collection fibers;

FIG. 4A is an end perspective view of a multi-probe imaging array of the subject matter described herein, comprising a round sample-imaging array interface;

FIG. 4B is an end perspective view of the multi-probe imaging array of FIG. 4A showing the placement of set screws;

FIG. 5A is a photograph of a multi-probe imaging array of the subject matter disclosed herein as it is being brought into proximity with an ex vivo biological sample;

FIG. 5B is a diagram of a multi-probe imaging array of the subject matter disclosed herein in contact with a biological sample present inside a biological sample containment and illumination apparatus;

FIG. 6A is an exploded view of an exemplary biological sample containment and illumination apparatus;

FIG. 6B is a perspective view of the exemplary biological sample containment and illumination apparatus described in FIG. 6A;

FIG. 7A is an end view of a multi-probe imaging array according to an embodiment of the subject matter described herein, showing a sample-imaging array interface surface having an array of 6×6 single channel fiber optic probe ends;

FIG. 7B is an end view of an illumination adapter according to an embodiment of the subject matter described herein;

FIG. 7C is an end view of a collection adapter according to an embodiment of the subject matter described herein;

FIG. 8A is an end view of a multi-probe imaging array according to an embodiment of the subject matter described herein, showing a sample-imaging array interface surface having an array of 10×10 single channel fiber optic probe ends;

FIG. 8B is an end view of an illumination adapter according to an embodiment of the subject matter described herein;

FIG. 8C is an end view of a collection adapter according to an embodiment of the subject matter described herein;

FIG. 9A is a schematic illustration of an imaging map according to an embodiment of the subject matter described herein;

FIG. 9B is a schematic illustration of an imaging map according to an embodiment of the subject matter described herein; and

FIG. 10 is a flow chart of an exemplary process for testing a biological sample according to an embodiment of the subject matter described herein.

### DETAILED DESCRIPTION

All references cited herein are incorporated herein by reference in their entireties to the extent that they supplement, explain, provide a background for, or teach methodology, techniques, and/or compositions employed herein.

Reference will now be made in detail to exemplary embodiments of the present subject matter, one or more examples of which are shown in the figures. Each example is provided to explain the subject matter and not as a limitation. In fact, features illustrated or described as part of one embodiment can be used in another embodiment to yield still a further embodiment. It is intended that the present subject matter cover such modifications and variations.

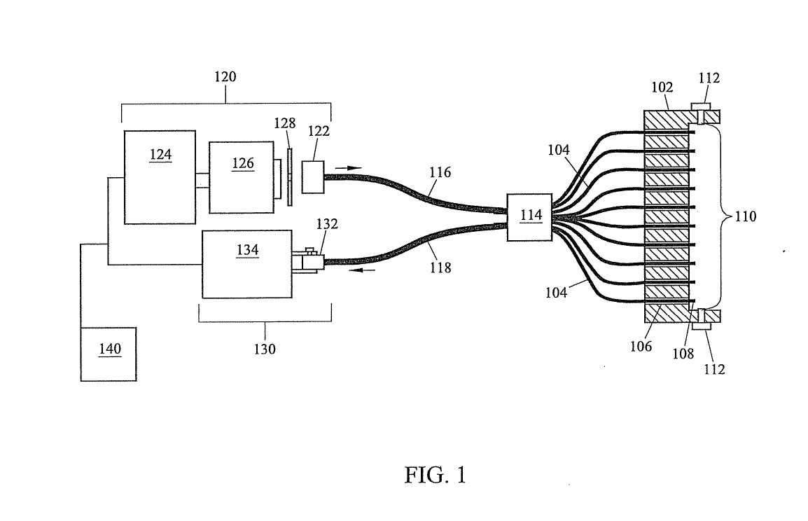

The subject matter disclosed herein includes a multi-probe imaging array for simultaneously irradiating multiple locations on a biological sample with electromagnetic radiation and detecting the absorbance or reflection of that radiation as an indication of a physical property of the biological sample. The array includes a probe holder to orient the plurality of probes such that there is no cross-talk between probes. FIG. 1 is a block diagram illustrating exemplary components of a system for imaging a sample using a multi-probe imaging array. Referring to FIG. 1, the system includes a multi-probe imaging array that includes probe holder 102 for holding and orienting plurality of probes 104 with respect to each other and with respect to a sample. Probe holder 102 can comprise any suitable material, such as a metal or hard plastic. Probe holder 102 can include a plurality of channels 106, into which the plurality of probes 104 are inserted. Ends 108 (which can also be referred to as imaging tips) of probes 104 extend past the surface of probe holder 102 to form sample-imaging array interface surface 110 wherein the ends 108 can be brought into contact with or be put into close proximity with a biological sample such that ends 108 illuminate the sample and detect absorption or reflection of the radiation. In some embodiments, probe holder 102 can include one or more adapter 112 for attaching the multi-array probe to a sample containment and illumination apparatus. Suitable adapters include screws and/or overhanging edges.

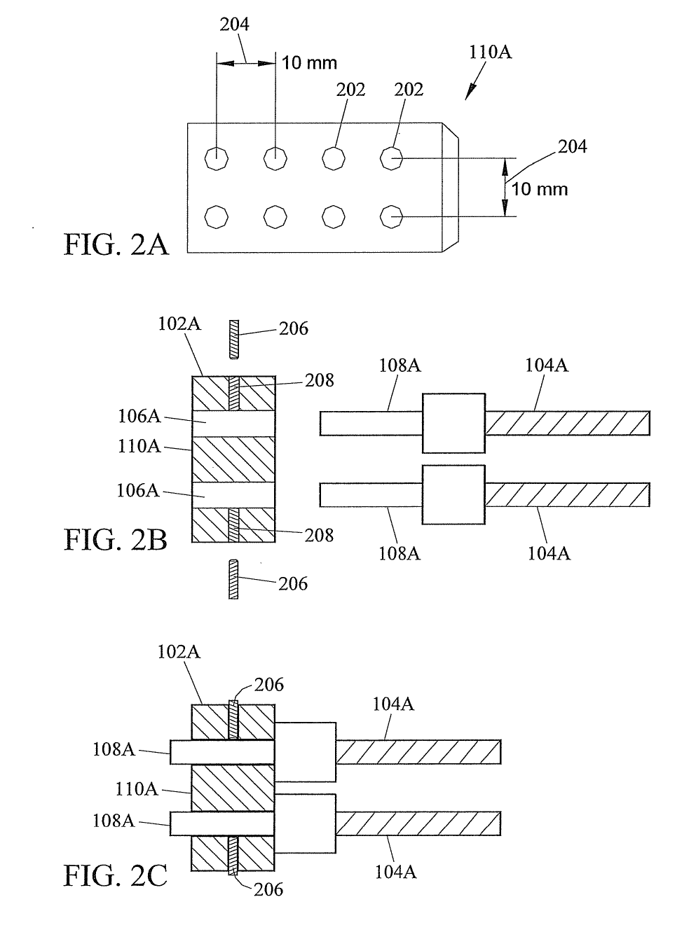

The 2-dimensional shape of the sample-imaging array interface surface can be any suitable shape, including, but not limited to rectangular, square, round or oval. In one implementation, the multi-probe imaging array can have a rectangular sample-imaging array interface surface. FIGS. 2A-2C illustrate different views of a multi-probe imaging array having a rectangular sample-imaging array interface surface. FIG. 2A illustrates a front perspective view of the multi-probe imaging array that shows sample-imaging array interface surface 110A. Surface 110A includes a four-by-two array of holes 202 such that the multi-probe imaging array can hold eight probes. The sample-imaging array interface surface can be designed to hold any number of probes desired. For example, sample-imaging array interface surfaces can be designed to have between 8 and 100 holes. Holes 202 can have center-to-center distance 204 of about 10 mm. For use with fiber optic probes as described further hereinbelow, holes 202 can have a diameter of, for example, about 3.75 mm. FIG. 2B shows a sectional view of the multi-probe imaging array with probes 104A separated from probe holder 102A. Ends 108A of probes 104A are insertable into channels 106A of probe holder 102A. The probe holder can include set screw channels 208, perpendicular to channels 106A, into which set screws 206 can be inserted to hold ends 108A in place within probe holder 102A, if desired. FIG. 2C shows a sectional view of the multi-probe imaging array with probes 104A in place in probe holder 102A. Ends 108A extend slightly from surface 110A. Set screws 206 have been inserted into set screw channels 208.

In some embodiments, probes 104 of FIG. 1 can be fiber optic probes. In some embodiments, the fiber optic probes can comprise fiber bundles comprising a plurality of fibers. In some embodiments, probes 104 comprise fiber bundles that include at least one illumination fiber for irradiating a location on a biological sample with electromagnetic radiation and at least one collection fiber for detecting the reflection or emission of electromagnetic radiation from the sample, for example due to the presence of fluorophores in the sample. Some fibers can be both illumination and collection fibers. The electromagnetic radiation can comprise radiation at one or more wavelengths between 250 and 900 nm. In some embodiments, the electromagnetic radiation can be visible light, at one or more wavelengths, or within one or more wavelength ranges, between 350 nm and 700 nm.

The fiber bundle can comprise a central core comprising one or more collection fibers surrounded by one or more symmetric rings of illumination fibers. For example, the fiber bundle can comprise one collection fiber surrounded by a ring of 2-8 illumination fibers. Alternatively, the fiber bundle can comprise a central core comprising one or more illumination fibers surrounded by one or more symmetric rings of collection fibers.

In some embodiments, the fiber bundle comprises an additional material or materials to act as a spacer in the fiber bundle. In some embodiments, the additional material is an absorptive material. In some embodiments, the additional material comprises additional fibers. Thus, the fiber bundle can further comprise one or more dead fibers for filling space within the fiber bundle. The dead fibers can fill space between collection or illumination fibers in a symmetric ring of collection or illumination fibers. Dead fibers can also form one or more rings surrounding the collection and illumination fibers.

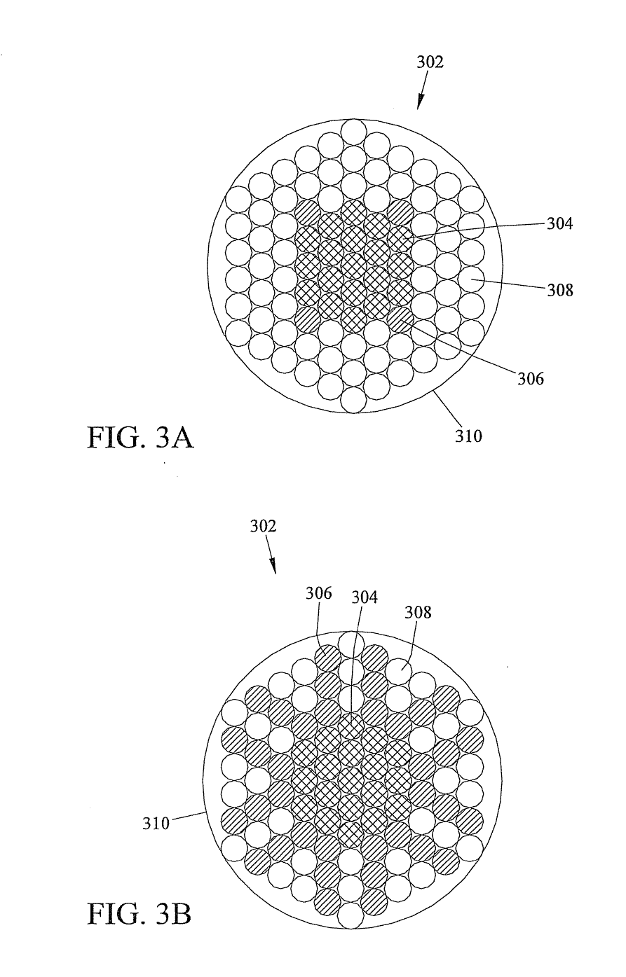

FIGS. 3A and 3B illustrate examples of fiber bundle geometries suitable for use in embodiments of the subject matter described herein. More particularly, in FIG. 3A, an end view of a fiber bundle comprising a central core of nineteen illumination fibers 304 is shown by cross-hatching in each illumination fiber. Four collection fibers 306 concentrically surround illumination fibers 304. In the illustrated example, collection fibers 306 are shown by single hatching in each fiber. Fibers 308 are dead fibers (shown with no shading or cross hatching) that are not used for collection or illumination and are merely used to package the fibers in a bundle. In order to hold the fibers in a bundle, a sheath 310 surrounds fibers 304, 306, and 308.

FIG. 3B shows an end view of a fiber bundle comprising a central core of illumination fibers 304 (shown by cross hatching). Collection fibers 306 (shown with single hatching) concentrically surround illumination fibers 304 in three symmetric ring arrangements. Dead fibers 308 (shown with no cross hatching or single hatching) fill up the space between collection fibers 306 to fill in the space created inside sheath 310. In one aspect of probe 104, each illumination fiber 304 and collection fiber 306 can have core/cladding diameters of 200/245 μm and a numerical aperture (NA) of 0.22.

In some embodiments, the fiber bundle has an inner illumination core of illumination fibers 304 having a diameter of between about 1000 and 1200 μm. The entire fiber bundle can have an outer diameter of between about 1 mm and about 5 mm. In some embodiments, the fiber bundle can have an outer diameter of about 3 mm. In some embodiments, the fiber optic probes have a sensing depth of between about 1 and 5 mm. In some embodiments, the sensing depth is between about 1 mm and about 2 mm.

In operation, the tips of illumination fibers 304 are placed in contact with the surface of a biological sample. Illumination fibers 304 deliver light to the tissue surface. The light propagates through the tissue, and a fraction of the light propagating through the tissue volume exits the tissue surface as a diffuse reflectance signal. Collection fibers 306 collect a portion of the emitted light from the tissue surface. Electrical signals correlating to the collected light can be used to determine an indication of a physical property of the sample, as described further below. The geometry of the illumination and collection fibers (separation between the fibers and their corresponding diameters) and the optical properties (absorption and scattering) of the tissue through which the light propagates, define the optical sensing depth (see Pfefer et al. (2003) Optics Letters 28:120-122; Zhu et al. (2003) J Biomed Opt 8:237-247; Zhu et al. (2006) Lasers Surg Med 38:714-724). See also Palmer et al. (2006) Appl Opt 45:1072-1078.

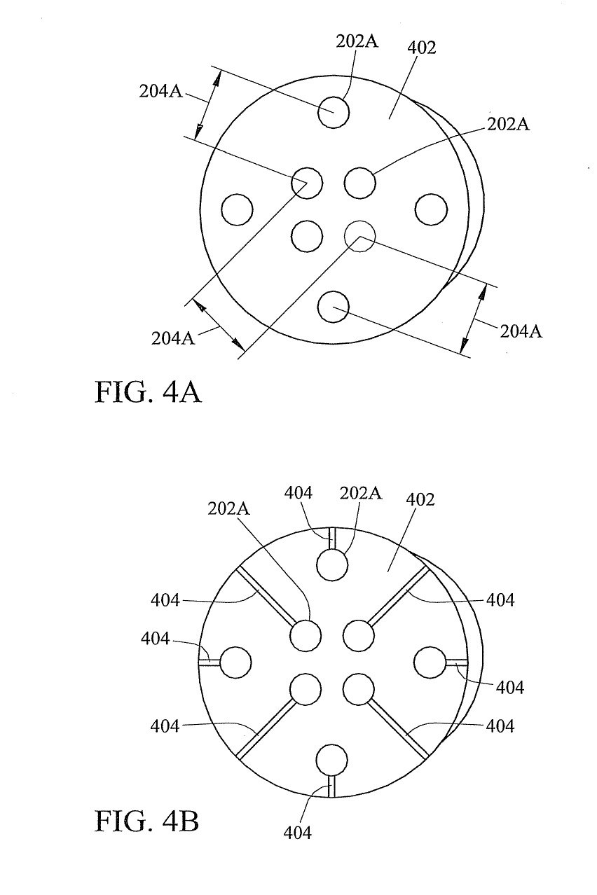

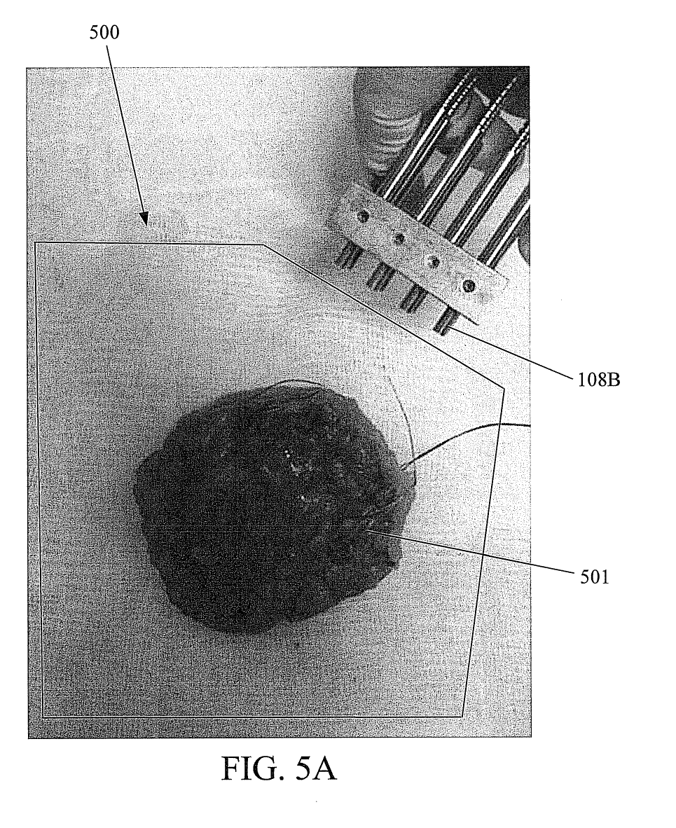

FIGS. 4A and 4B show end perspective views of a multi-probe imaging array having round sample-imaging array interface surface 402. As shown in FIG. 4A, surface 402 includes an array of eight holes 202A that have center-to-center distance 204A of about 10 mm. The array is arranged so that a grouping of four holes 202A are centered in the middle of surface 402. The four hole pattern is repeated at a 90 degree rotation, giving the full eight hole pattern shown. FIG. 4B shows round sample-imaging array interface surface 402 of FIG. 4A, showing the locations of holes 404 for the insertion of threaded set screws. Holes 404 are perpendicular to channels within the probe holder that correspond to probe holes 202A and can be used to insert set screws to hold probes in place, if desired.

Multi-probe arrays having sample-imaging array interface surfaces of any geometry can be used without a biological sample containment and illumination apparatus, for example, for freehand placement of the sample-imaging array interface surface against an ex vivo biological sample (e.g., an excised tumor) or an in vivo biological sample (either on the surface of a subject (e.g., a superficial tumor) or inside a body cavity (e.g., a surgical cavity, an oral cavity, or a cervical cavity) of a subject). In some embodiments, multi-probe imaging arrays comprising round or oval sample-imaging array interface surfaces can be used in methods involving the insertion of a multi-probe imaging array into a body cavity. The multi-probe imaging array can further comprise a protective cover mounted over the sample-imaging array interface surface to protect the probe ends from contamination with bodily fluids during the contacting with the biological sample. The protective cover should be translucent or transparent to allow for electromagnetic radiation to pass between the probe and the sample. The protective cover can be removable so that it can be cleaned between uses or replaced, for example, if the cover is disposable and intended for a single use. The protective cover can be formed of a material such as a biocompatible polymer.

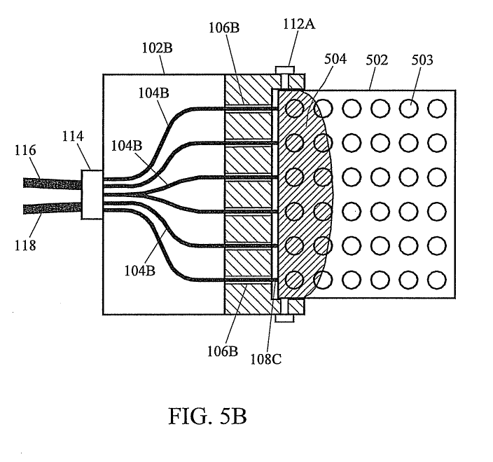

FIG. 5A is a photograph showing a multi-probe imaging array being moved into place for contacting an ex vivo biological sample that is not contained within a sample containment and illumination apparatus. In FIG. 5A, probe ends 108B are being moved by hand close to ex vivo biological sample 501 which is sitting on surface 500 of a sheet of absorptive paper. FIG. 5B shows an embodiment wherein a multi-probe imaging array is used to contact a biological specimen inside a biological sample containment and illumination apparatus. In some embodiments, multi-probe imaging arrays comprising rectangular or square sample-imaging array interface surfaces (such as that shown in FIGS. 2A-2C) can be used with a biological sample containment and illumination apparatus, such as those describe below, having rectangular or square sides or frame members. Referring to FIG. 5B, biological sample 504 (shown in single hatching going from lower left to upper right) is contained within biological sample containment and illumination apparatus 502, which comprises apertures 503 on one or more sides. Probe holder 102B comprises channels 106B surrounding probes 104B such that probe ends 108C extend from a sample-imaging array interface surface. When probe holder 102B is in contact with a side of biological sample containment and illumination apparatus 502, probe ends 108C can extend into apertures 503 on a side of biological sample containment and illumination apparatus 502 that faces the sample-imaging array interface surface. In some embodiments, ends 108C extend from the sample-imaging array interface surface and into apertures 503 such that ends 108C are flush with the inner surface of the side of the apparatus. In some embodiments, ends 108C are in direct contact with biological sample 504. In some embodiments, probe holder 102B comprises one or more adapter 112A (e.g., an overhang and/or a screw mechanism) for holding the probe holder onto a side of biological sample containment and illumination apparatus 502. FIG. 5B further shows fiber junction 114, illumination fiber bundle 116, and collection fiber bundle 118, which will be discussed in more detail below, with regard to FIG. 1.

Biological sample containment and illumination apparatus 502 may include a plurality of frame members positioned with respect to each other to form an interior space for receiving a biological sample. In one exemplary implementation, at least one of the plurality of frame members of biological containment and illumination apparatus 502 may be translatable with respect to another frame member such that the volume of interior space can be altered in at least one direction. In an alternate implementation of biological sample containment and illumination apparatus 502, at least two of the plurality of frame members are translatable in order to alter the volume of the interior space in at least two directions.

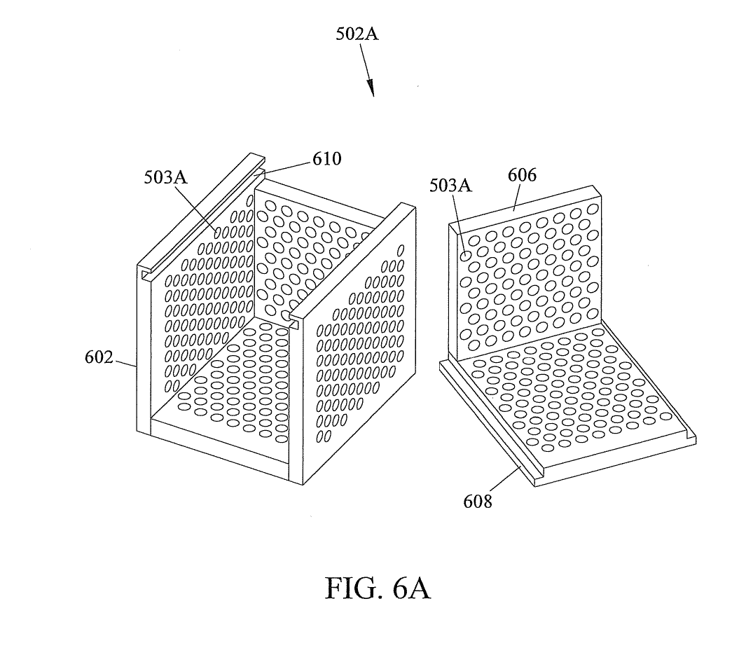

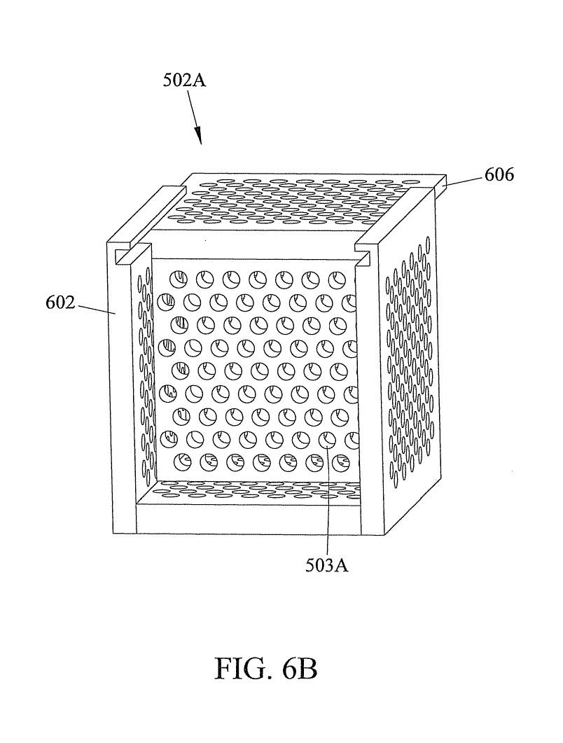

In one implementation, a biological sample containment and illumination apparatus can comprises a parallelepiped structure with at least two frame members that are moveable with respect to each other to vary the interior volume. FIGS. 6A and 6B illustrate different views of an exemplary biological sample containment and illumination apparatus according to an embodiment of the subject matter described herein. Referring to FIG. 6A, biological sample illumination and containment apparatus 502A includes a first frame member 602 and a second frame member 606. Frame member 606 is translatable with respect to frame member 602 to vary the interior volume of biological sample containment and illumination apparatus 502A. More particularly, frame member 606 includes a side 608 that slides in grooves 610 of frame member 602. Frame members 602 and 606 are separable from each other to facilitate cleaning and placement of biological samples. FIG. 6A is an exploded view of frame members 602 and 606 illustrating their separability. One or more frame members of biological sample containment and illumination apparatus include one or more probe receiving locations to allow for testing of a biological sample at plural locations on the sample without moving the sample. As illustrated in FIG. 6A, each side of frame member 602 as well as each side of frame member 606 includes apertures 503A for receiving probes. FIG. 6B shows frame members 602 and 606 placed together to form an interior space for holding a biological sample. Frame member 606 has been slid back in relation to the front-facing side of apparatus 502A in the slide of frame member 602 to create a smaller than maximal interior space. All sides of apparatus 502A includes apertures 503A.

The subject matter described herein is not limited to providing a biological sample containment and illumination apparatus where one frame member is translatable with regard to another frame member. In an alternate implementation, the frame members of biological sample containment and illumination apparatus may be fixed with respect to each other to define a fixed volume. In order to test biological samples of different volumes, biological sample containment and illumination apparatuses of different interior volumes can be provided.

Biological sample containment and illumination apparatus 502 may be made of any suitable material. In order to allow a user to view the biological sample when placed inside of apparatus 502, the material may be transparent or translucent. In one implementation, biological containment and illumination apparatus 502 may be made of an acrylic glass material, such as polymethyl methacrylate.

The translatable frame member or members of biological sample containment and illumination apparatus 502 can be employed to alter the volume of the interior space so that it approximates the volume of the biological sample. For example, the biological sample can be placed within the interior space and allowed to settle to an initial position by gravity. The position of one or more translatable frame members can then be changed such that at least a portion of each frame member is in contact with a portion of the biological sample. If desired, the shape of the biological sample can be forced to conform to the shape of the interior space by using the translatable frame members to apply a pressing force to the biological sample. In some embodiments, at least two frame members are translatable such that the volume of the interior space can be altered in at least two dimensions.

Apertures 503 of biological containment and illumination apparatus 502 can be spaced to correspond to the spacing of probe ends on a sample-imaging array interface surface of a multi-probe imaging array. In one exemplary implementation, each of apertures 503 has a diameter of about 3.7 mm and adjacent apertures have a 2-5 mm radial separation.

Referring once again to FIG. 1, probes 104 can be joined at fiber junction 114, where the fiber optic bundle of each probe is separated so that the collection fiber or fibers of each of probes 104 is bundled into a collection fiber bundle 118 and the illumination fiber or fibers of each of probes 104 is bundled into a illumination fiber bundle 116. Illumination fiber bundle 116 is joined from fiber junction 114 to illumination source 120. In one implementation, illumination source 120 can comprise a light source, such as a broadband light source and one or more filters (e.g., bandpass filters), spectral dispersion elements, or a monochromator to separate the light into particular wavelengths. In some embodiments, the light source comprises a white light source and no filters, so that the light is provided as a range of wavelengths (e.g., the range between about 350 nm and about 700 nm). In an alternate implementation, illumination source 120 can comprise a non-visible-light source, such as an infrared radiation source for illuminating a biological sample with infrared radiation. In yet another alternate implementation, illumination source 120 can include different narrowband light sources, such as light-emitting diode (LED) light sources for illuminating a biological sample with different wavelengths.

In the embodiment illustrated in FIG. 1, illumination source 120 includes xenon lamp 124 or another electromagnetic radiation source, such as an incandescent lamp, a white LED, a series of LEDs, or any other source of visible light. Light from xenon lamp 124 is coupled into illumination fibers of illumination fiber bundle 116 via illumination adapter 122. In the embodiment shown in FIG. 1, the different wavelengths of light are separated into single wavelength light or focused. Thus, illumination source 120 includes additional elements such as monochromator 126 and slit 128. White light from xenon lamp 124 can pass through the monochromator and slit and then be coupled into illumination adapter 122. The monochromator can be replaced by bandbass filters or a liquid tunable filter. In some embodiments, elements 126 and 128 are not present and 124 is coupled directly to 122.

Collection fiber bundle 118 is joined to detector 130 through collection adapter 132. Detector 130 can detect the emitted or reflected light signals detected by the collection fibers of probes 104 and output electrical signals corresponding to the optical signals. In one implementation, detector 130 can comprise a charge coupled device (CCD) camera and imaging optics 134. Detector 130 and illumination source 120 can be in communication with processor 140, (e.g., a laptop or other computer), which can be used to control illumination source 120, collect electrical signals from detector 130, convert the optical signals into values relating to physical properties of the biological sample, and output the values to a user. In some embodiments, detector 130 further comprises a radiation filtering device or spectral dispersion element, such as a diffraction grating, for example, coupled between a CCD camera and/or imaging optics and collection adapter 132. In particular, it can be desirable to include a radiation filtering device in detector 130 when no filters are used in illumination source 120 and the illumination source is a source that provides a broad range of wavelengths.

In order to account for the effects of probe geometry, and separate the effects of the absorbers, scatterers and fluorophores, processor 140 can run one or more simulations to generate a model of reflectance and then a model of fluorescence and then use the model to generate an indication of a physical property of the biological sample. Processor 140 can receive as input simulation start parameters, an initial guess of the optical properties of the biological sample into the model of reflectance. In one implementation, these optical properties may include absorption coefficient, scattering coefficient, anisotropy factor, and refractive index. An iterative inversion scheme can be used to optimize the fits to retrieve the actual optical properties of the biological sample. These optical properties can be used as inputs into the model of fluorescence to extract the intrinsic fluorescence. If the indication of intrinsic fluorescence to be determined is the concentration of one or more fluorophores, processor 140 can receive as input fluorophore characteristics, such as the extinction coefficient of the fluorophore at the excitation wavelength, the probability that a photon absorbed by a fluorophore will generate fluorescence, and the spectral probability distribution of the generated fluorescence at the emission wavelength. If these properties of the fluorophore are determined, then the concentration of the fluorophore in the biological sample can be determined. If these properties and not provided as input to processor 140, the indicator of intrinsic fluorescence may be an alternate measure of intrinsic fluorescence, such as the product of the quantum yield and the fluorophore concentration.

Monte Carlo modeling techniques (e.g., the use of an inverse Monte Carlo reflectance and fluorescence algorithm to convert electromagnetic radiation signals) can be used to design an optical probe that has a sensing depth of between about 1 mm and about 5 mm. In some embodiments, the probe has a sensing depth of between about 1 and about 2 mm within breast tissues. The Monte Carlo model allows simulation of light transport within a theoretical tissue model for different optical probe geometries and outputs a number of relevant parameters including the total signal detected by the probe (which will give a measure of signal to noise) as well as the distribution of the light-photons within the tissue model (i.e., optical sensing depth). Representative Monte Carlo modeling techniques for this purpose are disclosed in co-pending U.S. patent application Ser. No. 11/725,141 (corresponding to U.S. Patent Publication No. 2007/0232932), entitled “MONTE CARLO BASED MODEL OF FLUORESCENCE IN TURBID MEDIA AND METHODS AND SYSTEMS FOR USING SAME TO DETERMINE INTRINSIC FLUORESCENCE OF TURBID MEDIA,” filed Mar. 16, 2007, and co-pending U.S. patent application Ser. No. 11/119,865 (corresponding to U.S. Patent Publication No. 2006/0247532) entitled “METHOD FOR EXTRACTION OF OPTICAL PROPERTIES FROM DIFFUSE REFLECTANCE SPECTRA,” filed May 2, 2005, the disclosures of each of which is hereby incorporated by reference in its entirety.

In some embodiments, tens to hundreds of single-channel probes can be built into a multi-probe imaging array. Compared to a single-probe device, the multi-probe array can significantly increase the speed of analyzing a biological sample (which can be particularly useful during intra-operative tumor margin assessment, for example). The spatial resolution of an imaging system such as that shown in FIG. 1 is determined by the channel density of the probes, which is limited by cross-talk between adjacent probes due to tissue scattering. Photons from the illumination fibers of one channel can experience multiple scattering, propagate beyond the area with that channel and can be collected by collection fibers of another channel. Cross talk can be determined by several factors, including channel spacing, the illumination and collection geometry, and the tissue optical properties. In addition, the number of collection fibers for the imaging probe is based on the number of channels, number of pixels in the CCD, pixel size, magnification of the imaging optics and fiber size.

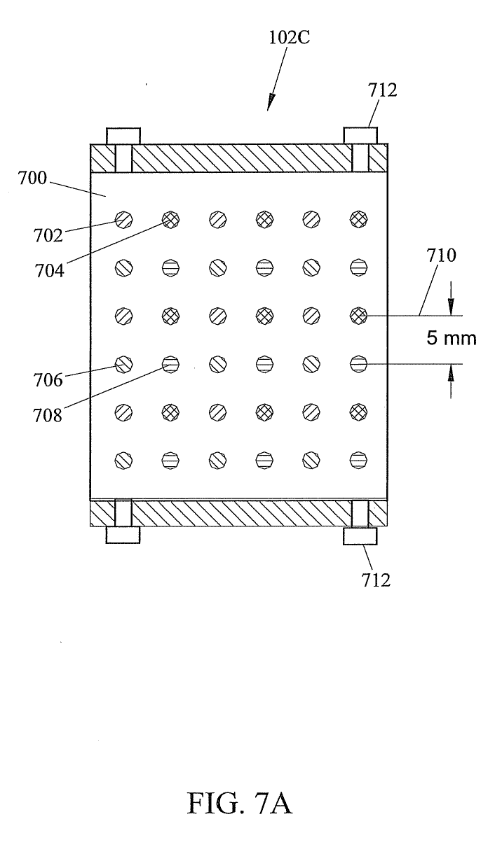

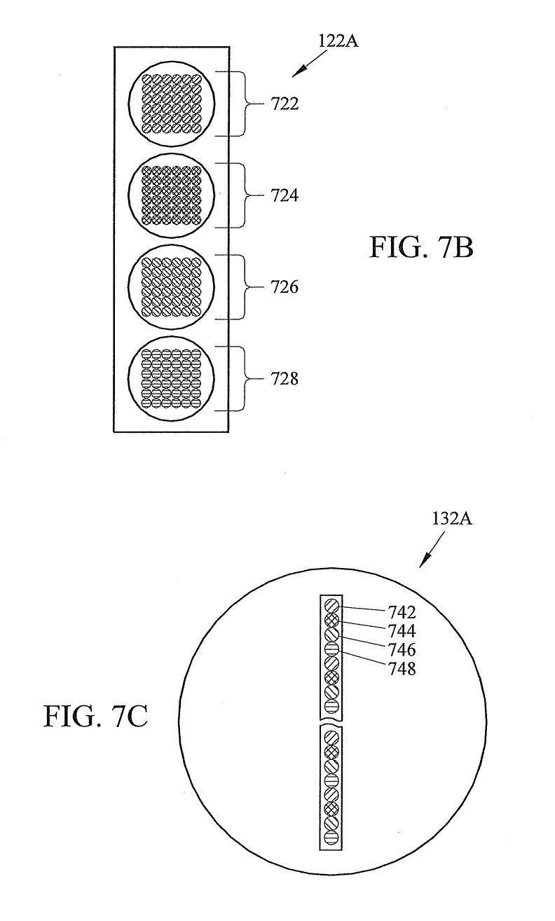

In one exemplary embodiment, in order to maximize channel density (the resolution of the imaging probe), without increasing cross-talk, probe holder 102C is designed so that single channel probes are arranged in a 6×6 pattern on sample-imaging array interface surface 700 according to channel type as shown in FIG. 7A. The top row of the array contains alternating probes in red channels 702 (shown in single hatching from lower left to upper right) and yellow channels 704 (shown in cross-hatching). The second row contains alternating probes in blue channels 706 (shown in single hatching going from upper left to lower right) and green channels 708 (shown in single hatching going from left to right). The alternating probe arrangement continues in the other rows of the array. On sample-imaging array interface surface 700, channel spacing 710 is 5 mm. As shown in FIG. 7A, sample-imaging array interface surface 700 includes four set screws 712 that can be used to hold sample-imaging array interface surface 700 to the face of a biological sample containment and illumination apparatus, if desired.

An end view of an illumination adapter 122A that can be used in an imaging system with sample-imaging array interface surface 700 shown in FIG. 7A is shown in FIG. 7B. At illumination adapter 122A, illumination fibers are bundled into four sections 722, 724, 726, and 728. Each section comprises illumination fibers from a fraction of the single channel probes. Section 722 consists of illumination fibers (shown in single hatching going from lower left to upper right) in the red channels. Section 724 consists of illumination fibers (shown in cross-hatching) in the yellow channels. Section 726 consists of illumination fibers (shown in single hatching going from upper left to lower right) in the blue channels and section 728 consists of illumination fibers (shown in single hatching from left to right) in the green channels. FIG. 7C shows the end view of collection adapter 132A which can be used in an imaging system that included sample-imaging array interface surface 700 of FIG. 7A. Collection fibers 742 (corresponding to red channels and shown in single hatching going from lower left to upper right), 744 (corresponding to yellow channels and shown in cross hatching), 746 (corresponding to blue channels and shown in single hatching going from upper left to lower right) and 748 (corresponding to green channels and shown in single hatching going from left to right) are bundled as shown. To reduce crosstalk on a CCD only one set or two sets of channels (red, yellow, blue and green) can be active.

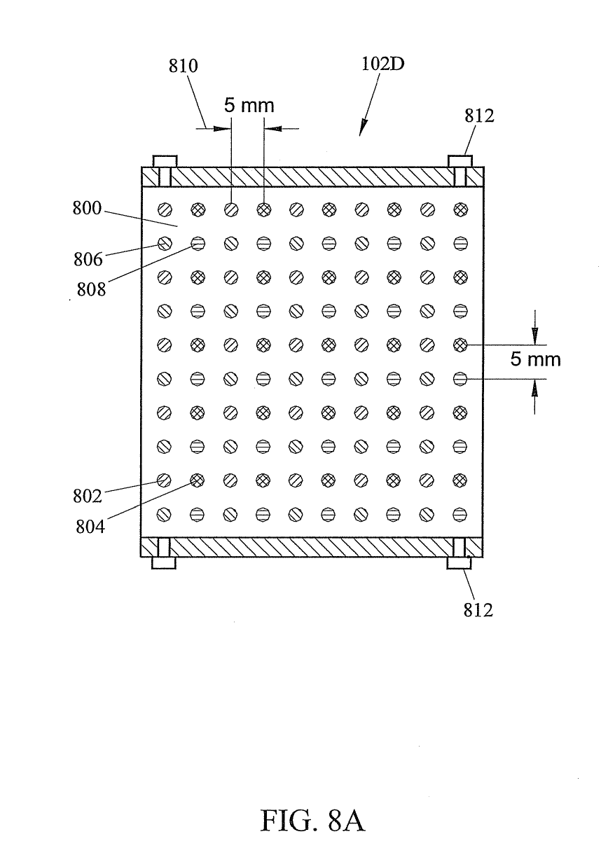

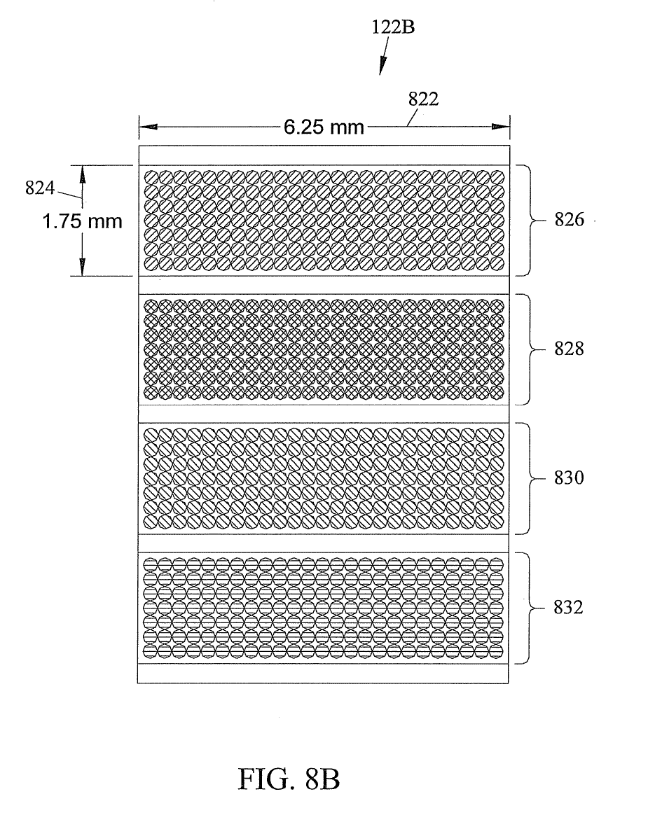

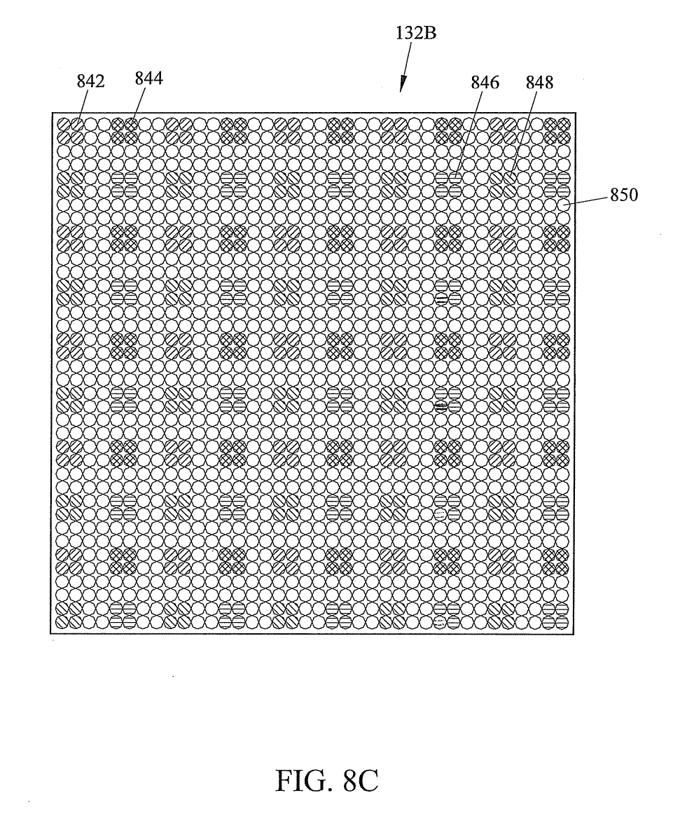

Another embodiment of a multi-probe array including a probe holder 102D having a sample-imaging array interface surface 800 with a 100 single channel probes, arranged in a 10×10 array, is shown in FIG. 8A. Alternate rows contain alternating probes in red channels 802 (shown in single hatching going from lower left to upper right) and yellow channels 804 (shown in cross-hatching) or in blue channels 806 (shown in singe hatching going from upper left to lower right) and green channels 808 (shown in single hatching going from left to right). On sample-imaging array interface surface 800, channel spacing 810 is 5 mm and sample-imaging array interface surface 800 includes four set screws 812 that can be used to hold sample-imaging array interface surface 800 to the face of a biological sample containment and illumination apparatus, if desired.

An end view of illumination adapter 122B that can be used in an imaging system comprising sample-imaging array interface surface 800 of FIG. 8A is shown in FIG. 8B. At illumination adapter 122B, illumination fibers are bundled into four sections 826, 828, 830, and 833, with each section having diameters 822 (6.25 mm) and 824 (1.75 mm). Section 826 consists of illumination fibers (shown in single hatching going from lower left to upper right) in the red channels. Section 828 consists of illumination fibers (shown in cross hatching) in the yellow channels. Section 830 consists of illumination fibers (shown in single hatching going from upper left to lower right) in the blue channels and section 832 consists of illumination fibers (shown in single hatching going from left to right) in the green channels.

For a 100 channel imaging probe, the maximum number of collection fibers per channel is less than or equal to 5 in order to be within the maximum number of collection fibers that can be imaged by a 512×512 CCD chip. FIG. 8C shows the end view of collection adapter 132B that can be used in an imaging system comprising sample-imaging array interface surface 800 of FIG. 8A. Collection fibers 842 (corresponding to red channels, and shown in single hatching going from lower left to upper right), 844 (corresponding to yellow channels and shown in cross hatching), 846 (corresponding to blue channels and shown in single hatching going from upper left to lower right) and 848 (corresponding to green channels and shown in single hatching going from left to right) are bundled as shown in groups of four fibers. Each collection fiber 842, 844, 846 and 848 is separated from any nearest neighbor collection fiber 842, 844, 846 and 848 of a different channel by as least two dead fibers 850 (shown as circles with no single hatching or cross hatching).

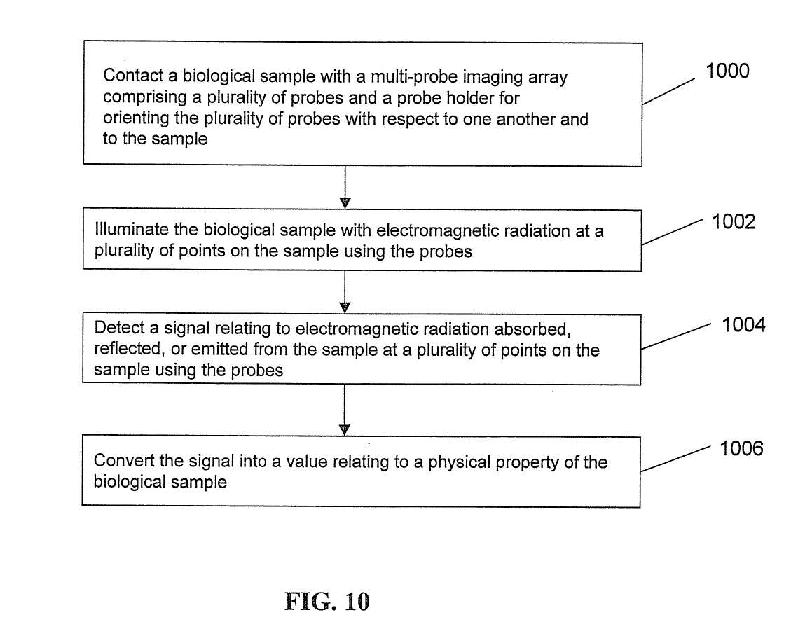

According to one aspect, the subject matter described herein includes a method for testing a biological sample. FIG. 10 is a flow chart of an exemplary method for testing a biological sample according to an embodiment of the subject matter described herein. Referring to FIG. 10, in step 1000 the sample-imaging array interface surface of the multi-probe imaging array is placed into proximity with a biological sample such that imaging ends of the multiple probe of the multi-probe array are in contact with plural locations on the biological sample and/or can irradiate the sample and detect reflection or absorption of the radiation from the sample.

As described above, the array can be contacted with an ex vivo biological sample or in vivo biological sample. The in vivo biological sample can be contacted by placing the multi-probe imaging array into a body cavity or onto the skin surface. The body cavity can be a surgical cavity. In one embodiment, the probe can be contacted to skin flaps during a skin saving breast cancer surgery to detect the presence or absence of malignant cells in the tissue of the skin flap, therefore providing the surgeon with information regarding whether or not additional tissue needs to be removed during the surgery. The probes can be placed into the oral cavity (e.g., in the oral mucosa or under the tongue). In particular, the in vivo uses of the presently disclosed subject matter can provide a rapid and non-destructive method to detect and quantify various analytes in biological tissues in real time. Thus, in vivo uses of the arrays can include, but are not limited to quantification of total hemoglobin, determination of blood loss, determination of the dilutional effect of fluid intake, and measurement of hemoglobin saturation in a tissue.

Alternatively, the biological sample can be excised from a subject and placed inside a biological sample containment and illumination apparatus. The sample-imaging array interface surface can then be placed in proximity to a side of the apparatus such that probes exposed on the surface or that extend from the surface can illuminate plural locations on the biological sample and detect emitted or absorbed light. In some embodiments, probes extending from the sample-imaging array interface surface extend into apertures in the side of the biological sample containment and illumination apparatus to contact the biological sample.

Irregardless of whether or not the biological sample is contained in a biological sample containment and illumination apparatus, is an in vivo sample, or is an ex vivo sample that is free of a biological sample containment and illumination apparatus, in the next step of the method, step 1002, a plurality of probes illuminate plural locations on the biological sample simultaneously. In step 1004, the probes detect reflected light or the absorption of light at the locations on the biological sample. The reflection or absorption of light can be converted into an electrical signal. In step 1006, the signal can be converted at a processor into a value relating to a physical property or parameter of the biological sample. Data processing in step 1006 can include, but is not limited to, one or more of the following: calibration of measured spectra, inversion to determine optical properties, calculation of absorber concentrations, and determination of relative fluorophore contributions and/or reduced scattering parameters. In some embodiments, the data is used to determine a property or parameter of the biological sample related to a biomarker concentration, hemoglobin saturation, a scattering coefficient, or a change in contrast in response to the presence of a contrast agent. The property or parameter can be referred to as a “value.” The biomarker concentration can include, but is not limited to, the concentration of oxyhemoglobin, deoxyhemoglobin, beta carotene, NADH, FAD, collagen, a porphyrin, elastin, keratin, tryptophan, or retinol.

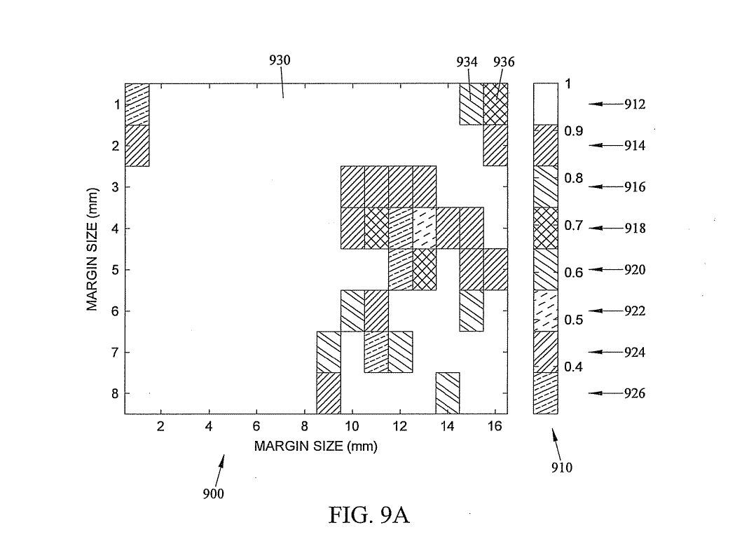

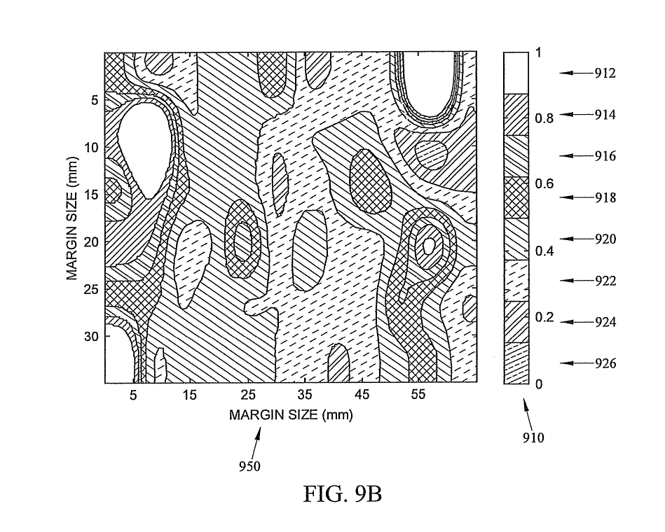

The values of each single measurement corresponding to each of the individual locations on the sample can be outputted in any convenient form (e.g., table, graph, etc). In some embodiments, processing step 1006 can include reconstruction of the computed values into images that can be outputted to a user. In some embodiments, the image can be a parameter map image of the biological sample wherein the value measured at a particular point on the biological sample is provided as a particular symbol or color to make user analysis of the data more efficient. Thus, in some embodiments, the method can provide an outputted 2-dimensional parameter map image that corresponds to multiple single measurements from a biological sample (e.g., from a tumor margin face), after optical spectra at each pixel are fed through a Monte Carlo model and optical properties and the concentration of absorbers in the tissue are quantified. FIG. 9A shows an exemplary parameter map image 900 where parameter values are presented as colors: dark red 912 (shown with no single hatching or cross hatching), pink 914 (shown with single hatching going from lower left to upper right), orange 916 (shown with double hatching going from upper left to lower right), yellow 918 (shown with cross hatching), green 920 (shown with single hatching going from upper left to lower right), light blue 922 (shown with single dashed hatching going from lower left to upper right), medium blue 924 (shown with double hatching going from lower left to upper right), or dark blue 926 (shown with hatching having alternating solid and dashed lines and going from lower left to upper right). Legend 910 shows colors 912, 914, 916, 918, 920, 922, 924, and 926 in order ranging from colors for values corresponding to the lowest probability of disease at the top to colors for values corresponding to the highest probability of disease at the bottom. Thus, in the parameter map image 900, area 930 is an area on the sample that has a low value (e.g., a low probability of disease), while areas 934 and 936 have medium values (e.g., medium probabilities for disease). FIG. 9B shows an exemplary parameter map image 950 with interpolation applied to remove pixelation. Again, values for each measurement have been converted to colors: dark red 912, pink 914, orange 916, yellow 918, green 920, light blue 922, medium blue 924, and dark blue 926 as indicated in legend 910 to the right of image 950. The values illustrated in images 900 and 950 can correspond to, for example, mean reduced scattering coefficient; the concentration of oxyhemoglobin, deoxyhemoglobin, beta carotene, or total hemoglobin; hemoglobin saturation % (which can be calculated from the equation [oxyhemoglobin]/[total hemoglobin]×100%); or any combination of such values (e.g., differences, sums, products, quotients (ratios), multivariate linear or nonlinear combinations, etc.) or to a probability prediction for the presence of cancer cells.

In some embodiments, the biological sample is treated with a contrast agent prior to contact with the multi-probe imaging array. For example, acetic acid is commonly used during colposcopic examination to identify atypical areas of the cervix that require biopsy. Addition of acetic acid in a concentration ranging from between about 3 and 6% causes acetowhitening of many cervical abnormalities including neoplasia, adenocarcinoma, and invasive squamous cell carcinoma. Without being bound to any one theory, the ability of acetic acid to cause whitening can be the result of acetic acid altering protein structure, which prevents light from passing through the epithelium, thereby enhancing light scattering. Using the presently disclosed multi-probe array and optical spectral imaging, acetic acid can be used as a contrast agent to detect many types of tumors, including detecting neoplastic tissue in breast tumor margins. The scattering from positive margins should be significantly greater than the scattering from negative margins, thereby providing contrast between malignant and non-malignant breast tissue. Thus in one embodiment, an excised tumor can be painted with 3-6% acetic acid and then imagined with an illumination source over the visible and near infrared wavelengths. Using a multi-probe imaging array, images from multiple points on the tumor margin can be captured over a period of between about 30-60 seconds, during which the acetowhitening decays. The images can capture the brightness and kinetics of the brightness to indicate whether the tumor margin is positive or negative. Additional contrast agents include other photosensitizers (e.g., amino levulinic acid (ALA), methylene blue, fluorescin, and other porphyrins). Still further contrast agents that could be painted onto biological samples for the detection of neoplastic tissue include acriflavin and 2-[N-(7-nitrobenz-2-oxa-1,3-dizaol-4-yl)amino]-2-deoxy-D-glucose (NBDG). NBDG is an optical analog of deoxyglucose used in PET imaging and is taken up with increased glucose metabolism. Acriflavin intercalates with DNA and can provide nuclear contrast. Acriflavin and NBDG could be detected by the probes of a multi-probe array as they fluoresce in the blue-green wavelengths.

### EXAMPLES

### (1) Monte Carlo Models

A Monte Carlo based inverse model, described in Palmer & Ramanujam (2006) Appl Opt 45:1062-1071, has been developed and employed to extract the absorption and scattering properties of breast tissues from measured diffuse reflectance spectra. More particularly, in the forward model, a set of absorbers are presumed to be present in the medium and the scatterer is assumed to be single sized, spherically shaped and uniformly distributed, which has been theoretically verified to be a reasonable assumption. See Palmer & Ramanujam (2006) Appl Opt 45:1062-1071. The wavelength-dependent absorption coefficients (μₐ(λ)) of the medium are calculated from the concentration of each absorber (Cᵢ) and the corresponding wavelength-dependent extinction coefficients (εᵢ(λ)), according to the relationship defined by Beer's law. The wavelength-dependent scattering coefficient (μₛ(λ)) and anisotropy factor (g) are calculated from scatterer size, density and the refractive index mismatch between the scatterer and surrounding medium using Mie theory for spherical particles. The absorption and scattering coefficients are then input into a scalable Monte Carlo model of light transport to obtain a modeled diffuse reflectance. The inversion process is achieved by adaptively fitting the modeled to the measured tissue diffuse reflectance. When the sum of the square of the errors between the modeled and measured diffuse reflectance are minimized, the absorption and scattering coefficients, the concentrations of absorbers, the scatter size and density are thereby extracted. The inversion takes several seconds on a Pentium 1.5 GHz computer.

The model was tested on homogeneous tissue phantoms, with optical properties representative of human tissues in the ultraviolet-visible spectral range. See Cheong, “Appendix to Chapter 8: Summary of Optical Properties” in Optical Theory Response of Laser-Irradiated Tissue” (New York: Plenum Press, 1995, pages 275-303). A set of 5 phantoms with similar scattering properties and a range of absorption properties (at a given wavelength) were tested. The wavelength-dependent extinction coefficients for hemoglobin were measured using a spectrophotometer. It was assumed that the oxygenation of hemoglobin was constant through the course of the experiment. The reduced scattering coefficient was determined from Mie theory, given the known size (1 μm), density, and refractive index of the spheres (1.60) and the surrounding medium, water (1.33).

In ex vivo studies (described in Zhu et al. (2006) Lasers Surg. Med. 38: 714-724 and Palmer et al. (2006) Applied Optics, 45: 1072-1078), a fiber-optic probe, a spectrometer and algorithms were used to quantify the concentration of the constituent absorbers (hemoglobin saturation, total hemoglobin content, and beta carotene concentration) and tissue scattering (a measure of cell morphology and density) in malignant, fibrous and adipose human breast tissues. Diffuse reflectance spectroscopy was carried out on 124 human breast tissues (54 malignant and 70 non-malignant) from 77 patients undergoing breast cancer surgery. Feature extraction from these measurements was carried out using the inverse Monte Carlo model. A Wilcoxon Rank Sum test was used to identify those features that show statistically significant differences between malignant and non-malignant breast tissues. These features were incorporated into a support vector machine algorithm (SVM), a binary classification algorithm based on statistical learning theory to classify a sample as malignant or non-malignant. Cross-validation techniques were used to test the performance of the algorithm in an unbiased manner.

Three of the four parameters extracted using the inverse Monte Carlo model showed statistically significant differences (p<0.001) between malignant and non-malignant tissues based on the Wilcoxon test. These included a statistically significant decrease in hemoglobin saturation and beta carotene concentration and a statistically significant increase in the wavelength averaged reduced scattering coefficient in malignant compared to non-malignant tissues. Hemoglobin saturation and the reduced scattering coefficient were input into SVM to determine the sensitivity and specificity for differentiating malignant vs. non-malignant breast tissues. The unbiased sensitivity and specificity for discriminating between malignant versus non-malignant breast tissues were 83% and 89%, respectively.

### (2) Intra-Operative Assessment of Breast Tissue Biopsy

The following example illustrates an application of the subject matter described herein for intra-operative assessment of a breast tissue biopsy.

Immediately after surgical excision, a biological sample comprising a breast tissue biopsy is dabbed dry and placed in biological sample containment and illumination apparatus. Adjustments are made by translating one or more frame members until the tissue conforms to the shape of biological sample containment and illumination apparatus and all 6 faces of the biological containment and illumination apparatus are flush with the biological sample. The sample-imaging array interface surface of a multi-probe array is aligned with apertures in one face of the biological sample and illumination apparatus such that exposed ends of fiber bundles of a plurality of probes are inserted into a plurality of apertures in the frame member of the biological sample containment and illumination apparatus until the ends of the fiber bundles are flush with the inner surface of the biological containment and illumination apparatus (e.g., in contact with the surface of the biological sample) and an optical measurement is made. Specifically, a diffuse reflectance spectrum over a wavelength range of 400-650 nm is recorded. This measurement takes less than a second to complete. This procedure is repeated for all 6 faces of biological sample containment and illumination apparatus.

The total measurement takes no more than a few seconds to a few minutes (e.g., less than 15 minutes) as compared to the tens of minutes used to take measurements at multiple locations on a sample using a single probe to take measurements at several locations sequentially. This can be beneficial both because it can reduce the time needed for margin assessment during surgery, thereby reducing the overall time of the surgery, but also because tissue can degrade somewhat overtime. Tissue diffuse reflectance spectra measured over 1 minute intervals have shown that there are minimal changes in diffuse reflectance (as well as in extracted parameters) over the course of four minutes. Over a 20 minute interval there is 7% decrease in blood saturation, a 23% increase in beta carotene concentration, and an 11% increase in the reduced scattering coefficient. Without being bound to any one theory, temporal degradation of tissue with regard to the variables which optical spectroscopy is sensitive to can be related to changes in oxygen transport or consumption post-excision or the drying of the tissue, which can result in changes in tissue volume and, hence, absorber concentration.

After the measurements are completed, several sites on each face of the biological containment and illumination apparatus are labeled with colored ink such that these sites can be evaluated by a pathologist for positive or negative margins. The number of sites that are marked with ink per specimen is coordinated with a pathologist. Because only a fraction of the margins would be expected to be positive, the majority of the margins evaluated are negative for disease. To increase the representation of disease in the collected spectral data set, each specimen can be cut in half after the margins have been optically examined and the spectra from known disease in the middle of the sample is measured and inked. In each patient, this site is considered to be representative of a positive margin since a positive margin is essentially malignant tissue extending all the way to the edge of the lumpectomy specimen.

A two-step algorithm is employed for classification of positive and negative margins: an inverse Monte Carlo model described in Palmer & Ramanujam (2006) Appl Opt 45:1062-1071 is employed for feature extraction (features related to breast tissue composition) from the diffuse reflectance spectra and a support vector machine classification algorithm (SVM) is used for classification based on these extracted features (see Zhu et al. (2006) Lasers Surg Med 38:714-724). The extracted features include absorbers and scatterers. The scattering coefficient is related to the size and density of scatterers present in the tissue (e.g., cell membranes, nuclei, structural proteins), whereas the absorption coefficient is related to the concentration of compounds present in tissue which absorb light in the visible wavelength regime (e.g., oxy- and deoxy-hemoglobin, beta carotene, and proteins). Negative margins can show the presence of clear alpha and beta bands of hemoglobin absorption (540 and 580 nm), which are only weakly present in a positive margin. The positive margin is expected to be less oxygenated and have more scattering than the negative margin. The data obtained can be verified by traditional pathology.

### (3) Training and Cross-Validation

The tissue composition parameters extracted from optical measurements of the specimens are grouped according to pathologic analysis of the margin status of the individually interrogated sites. Then, a classification algorithm is trained on these data in order to classify future optical tissue measurements as either “positive” or “negative” for cancer. This classification algorithm is formed by using a SVM algorithm for training, which is based on machine learning theory (Palmer et al. (2006) Appl Opt 45:1072-1078; and Gunn (1998) “Support Vector Machines for Classification and Regression” (University of Southampton, Department of Electronics and Computer Science), available at http://homepages.cae.wisc.edu/~ece539/software/svmtoolbox/svm.pdf). The classification algorithm is trained using a linear SVM based on the most discriminatory parameters; if the data do not support this, then various non-linear SVM algorithms are investigated to determine which gives the best classification performance for the clinical data. The sensitivity, specificity, and classification accuracy of the algorithm is then estimated using the leave-one-out cross validation method (Good (2001) Resampling Methods: A Practical Guide to Data Analysis, Birkhäuser, Boston, Mass., United States of America). In this method, one sample is removed from the training set, and the remaining data are used to train the SVM while the removed sample is used as a test sample to assess the classification accuracy of the algorithm. This provides an unbiased estimation of the sensitivity, specificity, and accuracy of the classification algorithm.

### (4) Predictive Model

In one embodiment, a model that provides a weighted combination of the diagnostically most significant extracted tissue parameters (from the Monte Carlo model) in the form of a computed index or probability distribution that maximizes the differences between positive and negatively diagnosed sites can be developed. Such a model can be based upon the parameters of a total of 2000 sites of know pathology. Since the outcome variable, pathologic diagnosis (1=positive, 0=negative), is dichotomous, the logistic regression model:

$$ln ( \frac{{p}_{i}}{1 - {p}_{i}} ) = α + {β}_{1} {x}_{1 , i} + {β}_{2} {x}_{2 , i} … {β}_{k} {x}_{k , i} i = 1 , 2 , … , 2000$$

can be used where x₁, x₂, . . . xₖ are the parameters (such as hemoglobin saturation and content, or the mean reduced scattering coefficient) extracted from diffuse reflectance spectra, and the coefficients β₁, β₂, . . . βₖ, are weights which describe the extent to which the corresponding tissue parameters contribute to differences between positive and negative pixels. The pᵢ are the predicted probabilities that a pixel is positive. Solving for the pᵢ, the equation becomes:

$${p}_{i} = \frac{1}{1 + {e}^{- ( α + {β}_{1} {x}_{1 , i} + {β}_{2} {x}_{2 , i} … {β}_{k} {x}_{k , i} )}} i = 1 , 2 , … , 2000$$

In other words, a predictive model that can be used to calculate the probability that a pixel is tumor-positive can be developed.

The logistic regression model described above relates to the simple case where each unit of observation (pixel) is independent, and is given for demonstrative purposes only. In practice, the predictive model can be developed by using a repeated measures logistic regression model to regress known pathology on the tissue parameters. Unlike ordinary logistic regression, the repeated measures logistic regression model does not require that all units of observations (i.e., pixels) be independent. Instead a compound symmetry correlation among pixels within the same margin is assumed, as is another compound symmetry correlation between pixels in different margins (i.e., block diagonal compound symmetry). The complicated and unpredictable spread of tumor cells in a margin will make it very difficult to use a more sophisticated correlation structure among pixels from the same subject, although other options, for example, spatial covariance structures can be studied. The repeated measures logistic regression model can be fit by SAS's GLIMMIX procedure (SAS Institute, Cary, N.C., United States of America).

Much of the statistical methodology that can be used to derive the presently described predictive model has been previously described by Harrell in “Regression Modeling Strategies With Applications to Linear Models, Logistic Regression and Survival Analysis” (New York: Springer-Verlag, 2001). Two of the tissue parameter variables are retained for model construction. A summary of the methodology is as follows: to fit the regression model to the 2 predictors, loess plots are used to explore whether the functional relationship between the outcome and a predictor is linear, quadratic, dichotomous, or some other more complex form perhaps involving a restricted cubic spline. Having chosen a form for both predictors, all terms are put in the model. It is expected that both tissue parameter variables, whether represented in the model as 1 degree of freedom or as multiple degrees of freedom, will have small p-values. In any case, the model is re-evaluated only if either one of the variables do not achieve at least a p-value of 0.50. This very conservative criteria for removal of variables from the model, recommended by Harrell, helps prevent overfilling. The c-index, a measure of a model's predictive accuracy, can be used to quantify the model's ability to discriminate positive and negative pathology results. The c-index is identical to the area under a receiver operating characteristic (ROC) curve and is the probability of concordance between the predicted probabilities and the observed responses. The c-index for the repeated measures logistic regression model will be approximated by calculating (0.5*Somers D)+0.5, where Somers D is a measure of association based on the number of concordance and discordant pairs. The model will be considered to have good discrimination only if the c-index is greater than or equal to 0.80. The requirement of a c-index of at least 0.80 is important for studies whose primary aim is to develop a diagnostic procedure.

While the c-index is useful in showing that a model can discriminate between pixels with positive and negative pathology, it is useful to know that the model has not been overfit. Instead of using a split sample method, the model will be evaluated with the simple bootstrap. See Efron and Tibshirani, “An Introduction to the Bootstrap” (New York: Chapman & Hall, 1993). In addition, however, Efron's enhanced bootstrap can be employed (see Efron, (1983) Journal of the American Statistical Association 78: 316-331), which allows an estimate of “optimism” which in turn is used to calculate an estimate of the amount that the model was overfilled. Bootstrapping can result in some deflation of the c-index.

The result of the above is a predictive equation, determined from discrete sites of known pathology, which will be applied to all sites (pixels) of unknown pathology to compute the probability, at each pixel site, that the underlying tissue probed by the imaging device contains cancer. This will enable the reconstruction of images of the margins indicating where positive margin sites are probable. We note that, all other things being equal, the absolute magnitude of predictive probabilities can be affected by the percentage of probe-positive pixels and the percentage of path-positive pixels in the analysis. However, a pixel with a fixed value on x1, x2, etc. will have the same ranked order predicted value regardless of the percentage of probe-positive and path-positive pixels in the analysis. Therefore, in general it is not the absolute magnitude of the predictive probabilities that is of interest, but the comparison of probabilities across the units of observation.

### (5) Image Reconstruction

Different possible methods of image reconstruction and interpretation can be employed. In one embodiment, a system of the presently disclosed subject matter can be used as a “black box”—the system is used to image a sample and a computer indicates either (a) that a particular margin is positive for cancer and further removal of tissue is necessary, or (b) that the margin is otherwise free of disease. This system would not require any input or interpretation from the user. A “black box” system can be achieved by the development of an appropriate classification algorithm to “interpret” the probability maps generated for the margins surveyed in this study.

A derivative map can be constructed on the original probability map to bring out areas of rapid change in predicted probability, which can indicate the presence of a boundary between normal and diseased tissue. The rationale for this approach is that if a margin is positive, the path-positive pixels are expected to be focal or multi-focal (span 1 to 2 pixels at most) based on the general pathologist's experience. This derivative map can also be rendered as a colormap, with areas of higher rate of change are rendered lighter than areas of lower rate of change. Through visual inspections of the margins' maps, characteristics can be identified that discriminate path-positive margins from path-negative margins. These characteristics, or features, can be converted into random variables and tested in a margin-level logistic regression model to predict true pathology status. For example, if a cluster of high probabilities is surrounded by rapid change to lower probabilities, a dichotomous variable can be created that indicates whether or not such a cluster was observed in the margin.

The statistical development of the margin-level logistic model can proceed as with the model described above. Since only about 90 of 1200 margins are expected to be path-positive, the formula above indicates that, at most, 6 variables can be screened. Since a within-subject correlation of about 0.06 is expected (calculated by simulation), the number of variables can be reduced to 5. As above, a model can be considered to be a good discriminator of pathology status if the c-index were greater than 0.80. If the c-index is high enough, a cutpoint can be determined that yields high sensitivity and specificity for classifying margin pathology status. For power calculations for the sensitivity rate, 90 (7.5%) of the 1200 margins from a total of 60 subjects are assumed to be pathology-positive, and that there will be little or no correlation within subject on margin classification status. The exact binomial test can be used to test the null hypothesis that the true sensitivity rate is 0.75 against the alternative hypothesis that the true sensitivity rate is 0.85. If at least 72 (82%) of the 90 margins are classified positive, then the null will be rejected. This test has one-sided alpha=0.07 and power=0.81. If exactly 82% of the margins are classified positive, then the exact 80% confidence interval will be 0.74-0.85. Margin classification according to computer algorithm will be compared to gold-standard margin-level pathology, and the sensitivity and specificity will be calculated.

As noted above, an advantage of developing a computer-aided classification algorithm is that diagnosis of margin status would not require user interpretation. However, a disadvantage is that this approach is more complex, and requires that features that indicate a positive margin must be explicitly defined and the algorithm must be trained to “recognize” them using a margin-level logistic regression model.

In addition to using computer algorithms to predict margin status based on margin surveillance images, an operating surgeon can predict the presence of positive margins based on their interpretation of processed images. This can be an important task, because the margin images obtained from the presently disclosed methods can have spatial features that are easily recognizable by the human observer, that are not otherwise considered in the computer automated classification algorithm. Humans are able to quickly extract features in an image and learn to recognize them in future images, something that is more challenging to program a computer to do, especially when the features are not well defined.

Thus, according to one embodiment, a subset of the 1200 margins previously assessed that had at least one pixel with confirmed histology is provided. For each of margins in the subset, the probabilities per pixel will be rendered as a color map such that pixels with higher probabilities will be rendered more red, and pixels with lower probabilities will be rendered blue. The color maps of 100 margins can be used to train surgeons to identify positive margins. The 100 margins can be deliberately selected so that half of the margins are path-positive and half are path-negative. Margins from different types of malignancy (ductal cancer, lobular cancer, and DCIS) can be included in the training images. For these training images, the surgeons can be shown the image, told whether the image belongs to a positive or negative margin, and given information about how positive areas are displayed on the colormap. After training the surgeons on 100 of the color maps, 100 new color maps can be used to test the surgeons' ability to identify positive margins. As with the training sample, these 100 can be selected to be half path-positive and to represent margins from each type of malignancy. For the training and testing images, cases can be selected in which the exact location of positive sites are validated by histology, and can include images which represent the range of normal and abnormal pixel values for both positive and negative margins. Margin classification according to surgeon will be compared to gold-standard margin-level pathology, and the surgeon's sensitivity and specificity will be calculated.

### (6) Establishing Number of Channels and Collection and Illumination Fibers

FIG. 1 shows a schematic of the main components of a system which consist of a xenon lamp, a monochromator, a slit, a multi-probe fiber optic imaging array, and a CCD camera with imaging optics. White light from the xenon lamp passes through the monochromator to split the light into its component wavelengths before the light is coupled into the illumination fibers via the illumination fiber adapter, and propagates to the distal end of the imaging probe. Diffuse reflectance from the tissue is detected by the collection fibers in each channel and propagates back to the collection fiber adapter, where it is imaged onto the CCD camera through imaging optics. Tens to hundreds of single-channel probes can be built into an imaging probe which can be used for 2-D measurements.

The majority of partial mastectomy specimens are smaller than 5 cm×5 cm×5 cm. Therefore, a mutli-probe imaging array with a sample-imaging array interface surface coverage of 5 cm×5 cm can be prepared. Within this area, the spatial resolution of the imaging system is determined by the channel density of the optical probes, which is limited by cross-talk between adjacent probes due to tissue scattering.

The number of collection fibers needed is based on the number of channels, number of pixels in a CCD, pixel size, magnification of the imaging optics and fiber size. The following calculations are applicable to a 512×512 CCD which is the same size as the 1024×256 CCD. The total number of fibers that can be imaged on the CCD is based on the following equation:

$$f = \frac{( {C}_{1} \times p - 4 d ) \cdot ( {C}_{2} \times p - 4 d )}{{( ( \frac{\sqrt{n} + 2}{\sqrt{n}} ) \times \sqrt{M} \times d )}^{2}}$$

Where, f is the total number of fibers, C₁ (1024) and C₂ (256) are the number of pixels in the vertical and horizontal direction, p is the pixel size (26 μm), n is the number of collection fibers per channel, M is the magnification of the system (1.1), and d is the fiber diameter (250 μm). The rule of thumb is to have at least a two fiber separation distance between each bundle of collection fibers to eliminate cross-talk on the CCD side and is represented by the “[sqrt(n)+2]/sqrt(n)]” part of the equation. The term “4d” in the equation allows for a 2 fiber dead space around the entire edge of the CCD. Table 1 shows the maximum number of collection fibers that can be imaged on the CCD chip where the number of collection fibers per channel is varied from 1-7.

TABLE 1
Maximum number of collection fibers that can be imaged onto the
CCD for different values of n.

| n | Maximum number of collection fibers on CCD |
|---|---|
| 1 | 224 |
| 2 | 320 |
| 3 | 450 |
| 4 | 480 |
| 5 | 561 |
| 6 | 583 |
| 7 | 660 |

A series of Monte Carlo simulations (see Wang et al. (1995) Computational Methods and Programs in Biomedicine 47: 131-146; Liu and Ramanujam (2006) Appl. Opt. 45: 4779-4790; and Zhu et al. (2005) J. Biomed. Opt. 10: 024032) have been carried out to evaluate the crosstalk for the multi-probe imaging array and determined that channel (i.e. probe) spacing of 10 mm achieves a signal to background of greater than 100 (1% cross-talk). With a channel spacing of 10 mm, a maximum of 25 channels can be built into a single imaging probe to cover a 5×5 cm area. In order to In order to maximize the channel density (the resolution of the imaging probe), without increasing crosstalk, sample-imaging array interface surface design was developed to increase the number of channels in the imaging probe by a factor of four, from 25 to 100 as shown in FIG. 8A. At the end of the probe that comes in contact with the tissue, the 100 channels are be arranged in a 10×10 pattern (FIG. 8A), resulting in a channel spacing, D=5 mm. At the illumination end (FIG. 8B), the illumination fibers are bundled into four sections: Section 1 will consist of all the illumination fibers in the red channels, Section 2 consists of those in the yellow channels, Section 3 consists of those in the blue channels, and Section 4 consists of those in the green channels in the imaging probe shown in FIG. 8A. Each section has a dimension of 1.75 mm by 6.25 mm (within the slit width and height), accommodating 175 fibers with diameter of 200/220/250 μm. For a 100 channel imaging probe, the maximum number of collection fibers per channel must be less than or equal to 5 in order to be within the maximum number of collection fibers that can be imaged by the CCD chip, which in this case is 500. Using a conservative estimate of 4 collection fibers per channel, the collection bundle (see FIG. 8C) is packaged in the same order as in the imaging probe. Dead fibers (in gray) are used between the channels.

### (7) Construction of An Optical Imaging System

Single-channel fiber optic probes can be obtained or prepared from a suitable commercial source (e.g., RoMack Inc., Williamsburg, Va., United States of America). A 450 W xenon lamp and a monochromator can be purchased (e.g. from HORIBA Jobin Yvon, Inc; Edison, N.J., United States of America). The exit slit of a monochromator can be modified so that the illumination adapter of the imaging probe can be easily aligned to the slit. A back-illuminated thermoelectrically cooled, 512×512 pixel UV/Visible CCD camera can be purchased and reflective imaging optics designed to image the fibers in the collection adapter onto the CCD chip with a 1× magnification.

An imaging probe array which consists of 100 individual channels can be prepared having a sample-imaging array interface surface of the orientation shown in FIG. 8A, in which the probes will extend out of the probe holder surface a fixed distance so that they can be inserted into the apertures of a plexi-glass biological sample containment and illumination apparatus. In order to interface the probe with a partial mastectomy specimen, the array can be aligned with each face of the plexi-glass biological sample containment and illumination apparatus, which can have dimensions of, for example, 5×5×10 cm, until the tip of the probes are flush with the inner surface of the apparatus. A bracket of set screws will be used to lock the sample-imaging array interface surface to the face of the biological sample containment and illumination apparatus before a measurement is made.

It will be understood that various details of the presently disclosed subject matter may be changed without departing from the scope of the presently disclosed subject matter. Furthermore, the foregoing description is for the purpose of illustration only, and not for the purpose of limitation.

1. A multi-probe imaging array for imaging a sample, the multi-probe imaging array comprising a plurality of electromagnetic radiation probes and a probe holder for orienting the plurality of probes with respect to one another and to the sample, wherein at least some of the probes simultaneously illuminate the sample and detect resulting electromagnetic radiation from the sample.

2. The multi-probe imaging array of claim 1, wherein the probe holder orients the plurality of probes with respect to one another to avoid cross-talk between probes.

3. The multi-probe imaging array of claim 2, wherein each of the plurality of electromagnetic radiation probes comprises a fiber optic probe, wherein the fiber optic probe comprises a fiber bundle comprising at least one illumination fiber and at least one collection fiber.

4. The multi-probe imaging array of claim 3, wherein the fiber bundle comprises one or more of:
a symmetric ring of illumination fibers surrounding a core of collection fibers or a symmetric ring of collection fibers surrounding a core of illumination fibers;
at least one dead fiber or an absorptive material;
a central illumination core comprising 19 illumination fibers symmetrically surrounded by four collection fibers and a plurality of dead fibers; and
a central collection fiber surrounded by a symmetric ring of between 2 and 8 illumination fibers.

5-11. (canceled)

12. The multi-probe imaging array of claim 3, wherein the probe holder comprises a series of channels and a sample-imaging array interface surface comprising a series of holes, wherein each of the series of holes corresponds to one end of each of the channels, and wherein a portion of each of the fiber bundles is enclosed within one of the channels such that a first end of the fiber bundle extends up to or beyond a hole on the surface of the probe holder such that the first end of the fiber bundle is free of the probe holder.

13-16. (canceled)

17. The multi-probe imaging array of claim 12, wherein the probe holder further comprises one or more set screws to hold a probe in place, wherein each of the one or more set screws is oriented in the probe holder perpendicular to one of the channels.

18. The multi-probe imaging array of claim 12, further comprising a fiber junction to which a second end of each of the fiber bundles is coupled, wherein the fiber junction is further coupled to a detector and to an illumination source, wherein the detector receives electromagnetic radiation signals from the probes and converts the electromagnetic radiation signals to electrical signals, and wherein the illumination source provides electromagnetic radiation to the probes.

19. The multi-probe imaging array of claim 18, wherein illumination fibers from each of the fiber bundles are bundled at the fiber junction to form an illumination fiber bundle, and wherein the illumination fiber bundle is coupled from the fiber junction to an illumination source.

20. The multi-probe imaging array of claim 19, wherein the illumination source comprises an electromagnetic radiation source and an illumination adapter.

21. The multi-probe imaging array of claim 20, wherein the illumination fiber bundle is separated at the illumination adapter into a plurality of sets, wherein each of the sets comprises illumination fibers that emit a common wavelength range from the plurality of probes.

22. (canceled)

23. The multi-probe imaging array of claim 20, wherein the electromagnetic radiation source comprises an incandescent lamp which is filtered through a series of bandpass optical filters, a spectral dispersion element, or a diffraction grating.

24. (canceled)

25. The multi-probe imaging array of claim 18, wherein collection fibers from each of the fiber bundles are bundled at the fiber junction to form a collection fiber bundle, and wherein the collection fiber bundle is coupled from the fiber junction to the detector.

26. The multi-probe imaging array of claim 25, wherein the detector further comprises a collection adapter for arranging collection fibers such that detector ends of collection fibers detecting the same wavelength range are not adjacent to each other to minimize cross-talk, wherein the collection adapter is coupled to an imaging device.

27. The multi-probe imaging array of claim 26, wherein the imaging device comprises a charge-coupled device (CCD).

28. The multi-probe imaging array of claim 27, wherein the imaging device comprises a spectral dispersion element, wherein the spectral dispersion element is coupled between the CCD and the collection adapter.

29-31. (canceled)

32. The multi-probe imaging array of claim 1, further comprising an adapter for attaching the multi-probe imaging array to a biological sample containment and illumination apparatus such that ends of at least some of the probes contact the sample through apertures in the biological sample containment and illumination apparatus.

33. The multi-probe imaging array of claim 1, wherein at least some of the probes simultaneously illuminate the sample with electromagnetic radiation at one or more wavelengths ranging between 250 nm and 900 nm.

34. A method for testing a biological sample, the method comprising:
contacting the biological sample with a multi-probe imaging array, wherein the multi-probe imaging array comprises a plurality of electromagnetic radiation probes and a probe holder for orienting the plurality of probes with respect to one another and to the sample;
illuminating the biological sample using the probes, wherein illuminating the sample comprises illuminating plural locations on the biological sample surface in parallel; and
detecting electromagnetic radiation reflected from or emitted by plural locations on the biological sample in response to the illumination.

35. The method of claim 34, wherein the probe holder orients the plurality of probes with respect to one another to avoid cross-talk between probes.

36. The method of claim 35, wherein each of the plurality of electromagnetic radiation probes comprises a fiber optic probe, wherein the fiber optic probe comprises a fiber bundle comprising at least one illumination fiber and at least one collection fiber.

37. The method of claim 36, wherein the fiber bundle comprises one or more of:
a symmetric ring of illumination fibers surrounding a core of collection fibers or a symmetric ring of collection fibers surrounding a core of illumination fibers;
at least one dead fiber or an absorptive material;
a central illumination core comprising 19 illumination fibers symmetrically surrounded by four collection fibers and a plurality of dead fibers; and
a central collection fiber surrounded by a symmetric ring of between 2 and 8 illumination fibers.

38-44. (canceled)

45. The method of claim 36, wherein the probe holder comprises a series of channels and a sample-imaging array interface surface comprising a series of holes, wherein each of the series of holes corresponds to one end of each of the channels, and wherein a portion of each of the fiber optic probes is enclosed within one of the channels such that a first end of the fiber bundle extends up to or beyond a hole on the surface of the probe holder such that the first end of the fiber bundle is free of the probe holder.

46-49. (canceled)

50. The method of claim 45, wherein contacting the biological sample with the multi-probe imaging array comprises placing the sample-imaging array interface surface against an outer surface of the biological specimen.

51. The method of claim 50, wherein the biological sample is located in a subject and is selected from the group consisting of a body cavity surface, a superficial tumor, and an undersurface of a skin flap created during a surgery.

52. The method of claim 51, wherein the subject is a human subject.

53. The method of claim 51, wherein the body cavity surface is in an oral cavity and wherein detecting electromagnetic radiation further comprises detecting a signal indicative of blood volume in the subject.

54. The method of claim 51, wherein the biological sample is a superficial tumor and wherein detecting electromagnetic radiation further comprises detecting a signal indicative of one or more of the group consisting of a response to a medical therapy, a lack of response to a medical therapy, the presence of cancer cells, and the absence of cancer cells.

55. The method of claim 51, wherein the body cavity surface is in open surgical cavity and wherein detecting electromagnetic radiation further comprises detecting a signal indicative of the presence or absence of cancer cells.

56. The method of claim 51, wherein the biological sample is a skin flap created during a skin-saving mastectomy surgery, and wherein detecting electromagnetic radiation further comprises detecting a signal indicative of the presence or absence of cancer cells.

57. The method of claim 50, wherein the multi-probe imaging array further comprises a protective cover mounted over the sample-imaging array interface surface to protect the probes from contamination by the biological sample.

58. (canceled)

59. (canceled)

60. The method of claim 45, wherein the method further comprises placing the biological sample in an interior space defined by a biological sample containment and illumination apparatus having a plurality of frame members, at least one of which includes a plurality of probe receiving locations for receiving a plurality of electromagnetic radiation probes and for positioning the probes with respect to the sample to allow illumination of plural locations of the sample by the probes.

61. The method of claim 60, wherein the biological sample comprises an excised tumor tissue.

62. The method of claim 60, wherein at least one of the plurality of frame members is translatable with respect to another frame member such that the volume of interior space can be altered in at least one direction.

63. (canceled)

64. (canceled)

65. The method of claim 60, wherein the probe receiving locations comprise a plurality of apertures adapted to receive fiber optic probes.

66. The method of claim 65, wherein contacting the biological sample with the sample-imaging array interface surface comprises contacting the sample-imaging array interface surface with an outer surface of a frame member of the biological sample containment and illumination apparatus and inserting a plurality of fiber optic probe tips into a plurality of apertures so that the probe tips are flush with an interior surface of the frame member.

67-71. (canceled)

72. The method of claim 34, wherein detecting electromagnetic radiation further comprises detecting a signal indicative of one or more of the group consisting of a biomarker concentration, a scattering coefficient, and a change in contrast in response to the presence of a contrast agent.

73. The method of claim 72, wherein detecting the signal indicative of one or more of the group consisting of a biomarker concentration, a scattering coefficient, and a change in contrast in response to the presence of a contrast agent comprises applying an inverse Monte Carlo reflectance and fluorescence algorithm to convert an electromagnetic radiation signals to one of the group consisting of a biomarker concentration, a scattering coefficient, and a change in contrast in response to the presence of a contrast agent.

74. The method of claim 72, wherein the contrast agent is selected from the group consisting of acetic acid, acriflavin, 2-[N-(7-nitrobenz-2-oxa-1,3-diazol-4-yl)amino]-2-deoxy-D-glucose (NBDG), and fluorescein.

75. The method of claim 72, wherein the biomarker concentration is selected from the group consisting of oxyhemoglobin concentration, deoxyhemoglobin concentration, beta carotene concentration, hemoglobin saturation, total hemoglobin concentration, NADH, FAD, collagen, porphyrin, elastin, keratin, tryptophan, or retinol.

76. The method of claim 72, further comprising preparing a parameter map image of the biological sample, wherein the image provides a value for a plurality of points on the biological specimen, wherein said value is a value related to one of the group consisting of mean reduced scattering coefficient, a biomarker concentration, hemoglobin saturation, and any combination thereof.

77. (canceled)

78. The method of claim 34, wherein the method is performed during a surgery to remove a tumor, and detecting electromagnetic radiation comprises providing tumor margin assessment.

79. A system for imaging a biological sample, the system comprising:
a multi-probe imaging array for simultaneously illuminating a plurality of locations of the biological sample with electromagnetic radiation generated by an electromagnetic radiation source and for detecting electromagnetic radiation reflected from or emitted by the biological sample, the multi-probe imaging array comprising a plurality of electromagnetic radiation probes and a probe holder for orienting the plurality of probes with respect to one another and to the sample;
a detector coupled to the multi-probe imaging array for receiving electromagnetic radiation signals indicative of the electromagnetic radiation detected by the probes and for converting the electromagnetic radiation signals to electrical signals; and
a processor coupled to the detector for receiving the electrical signals and for determining, based on the electrical signals an indication of a physical property of the biological sample.

80. The system of claim 79, wherein the probe holder orients the plurality of probes with respect to one another to avoid cross-talk between probes.

81. The system of claim 80, wherein each of the plurality of electromagnetic radiation probes comprises a fiber optic probe, wherein said fiber optic probe comprises a fiber bundle comprising at least one illumination fiber and at least one collection fiber.

82. The system of claim 81, wherein the fiber bundle comprises one or more of:
a symmetric ring of illumination fibers surrounding a core of collection fibers or comprises a symmetric ring of collection fibers surrounding a core of illumination fibers;
at least one dead fiber or an absorptive material;
a central illumination core comprising 19 illumination fibers symmetrically surrounded by four collection fibers and a plurality of dead fibers; and
a central collection fiber surrounded by a symmetric ring of between 2 and 8 illumination fibers.

83-89. (canceled)

90. The system of claim 81, wherein the probe holder comprises a series of channels and a sample-imaging array interface surface comprising a series of holes, wherein each of the series of holes corresponds to one end of each of the channels, and wherein a portion of each of the fiber bundles is enclosed within one of the channels such that a first end of the fiber bundle extends up to or beyond a hole on the surface of the probe holder such that the first end of the fiber bundle is free of the probe holder.

91-94. (canceled)

95. The system of claim 90, wherein the probe holder further comprises one or more set screws to hold a one or more probes in place, wherein each of the one or more set screws is oriented in the probe holder perpendicular to one of the channels.

96. The system of claim 90, further comprising a fiber junction to which a second end of each of the fiber bundles is coupled, wherein the fiber junction is further coupled to the detector and to an illumination source, wherein the illumination source provides electromagnetic radiation to the probes.

97. The system of claim 96, wherein illumination fibers from each of the fiber bundles are bundled at the fiber junction to form an illumination fiber bundle, and wherein the illumination fiber bundle is coupled from the fiber junction to an illumination source.

98. The system of claim 97, wherein the illumination source comprises an electromagnetic radiation source and an illumination adapter.

99. The system of claim 98, wherein the illumination fiber bundle is separated at the illumination adapter into a plurality of sets, wherein each of the sets comprises illumination fibers that emit a common wavelength range from the plurality of probes.

100. (canceled)

101. The system of claim 98, wherein the electromagnetic radiation source comprises an incandescent lamp which is filtered through a series of bandpass optical filters, a spectral dispersion element, or a diffraction grating.

102. (canceled)

103. The system of claim 96, wherein collection fibers from each of the fiber bundles are bundled at the fiber junction to form a collection fiber bundle, and wherein the collection fiber bundle is coupled from the fiber junction to the detector.

104. The system of claim 103, wherein the detector further comprises a collection adapter for arranging collection fibers such that detector ends of collection fibers detecting the same wavelength range are not adjacent to each other to minimize cross-talk, wherein the collection adapter is coupled to an imaging device.

105. The system of claim 104, wherein the imaging device comprises a charge-coupled device (CCD).

106. The system of claim 105, wherein the imaging device comprises a spectral dispersion element, wherein the spectral dispersion element is coupled between the CCD and the collection adapter.

107. (canceled)

108. The system of claim 79, further comprising a biological sample containment and illumination apparatus for holding a biological sample for illumination by the multi-probe imaging array, the biological containment and illumination apparatus comprising a plurality of frame members positioned with respect to each other to form an interior space for receiving a biological sample, wherein at least one of the plurality of frame members includes a plurality of probe receiving locations for receiving a plurality of electromagnetic radiation probes and for positioning the probes with respect to the biological sample to allow illumination of plural locations of the biological sample by the probes.

109. The system of claim 108, further comprising an adapter for attaching the multi-probe imaging array to the biological sample containment and illumination apparatus.

110. The system of claim 108, wherein at least one of the plurality of frame members is translatable with respect to another frame member such that the volume of interior space can be altered in at least one direction.

111-115. (canceled)
